(19) United States
(12) Patent Application Publication
(10) Pub. No.: US 2022/0275459 A1
(43) Pub. Date: Sep. 1, 2022

(54) TUMOUR MARKER AND USE THEREOF

(71) Applicant: Lisen Imprinting Diagnostics Wuxi Co., Ltd., Wuxi, Jiangsu Province (CN)

(72) Inventors: Tong Cheng, Wuxi (CN); Ning Zhou, Wuxi (CN)

(21) Appl. No.: 17/631,074

(22) PCT Filed: Jul. 28, 2020
(86) PCT No.: PCT/CN2020/105047

§ 371 Date: Jan. 28, 2022

(30) Foreign Application Priority Data
Jul. 30, 2019 (CN) 2019 10695955.6

(51) Int. Cl. C12Q 1/6886

(52) U.S. Cl. CPC C12Q 1/6886; C12Q 2600/136; C12Q 2600/154; C12Q 2600/158; C12Q 2600/112

(57) ABSTRACT

This application provides a tumor marker and its applications. The tumor marker includes any one, or a combination of at least two, of imprinted genes Z17, Z18, Z19, Z20, Z21, Z22, Z23, Z25, Z26, Z27, and Z28. If necessary, the tumor marker may further include any one, or a combination of at least two, of imprinted genes Z1, Z2, Z3, Z4, Z5, Z6, Z8, Z9, Z10, N11, Z12, Z13, Z14, Z15, and N16. The imprinted genes in this application have significant diagnostic sensitivity when used as tumor markers for tumor diagnosis and can therefore facilitate early diagnosis of tumors, help increase patients' survival rates, and thus contribute greatly to saving patients' lives. The imprinted genes can also provide important guidance on the treatment and medication of tumors.

### TECHNICAL FIELD

This application pertains to the field of biotechnology, in particular the field of genetic diagnosis technology, and relates to a tumor marker and its applications. More particularly, the application relates to the use of an imprinted gene-based tumor marker, of a tumor diagnosis model based on the imprinted gene-based tumor marker, or of a tumor diagnosis device based on the imprinted gene-based tumor marker in preparing a drug and/or a reagent for use in diagnosing, screening, or treating a tumor.

### DESCRIPTION OF RELATED ART

Malignant tumors are one of the major factors that pose a serious threat to human health. Each year there are 14 million new cases of cancer patients and 8.2 million deaths caused by cancer globally. Infinite proliferation and metastasis are the two major features of cancer cells, and most cancer patients die because the cancer cells metastasize rapidly and cause such complications as organ failure. Early cancer lesions are relatively small in size; therefore, not only do they result in relatively small surgical wounds when treated surgically, but also only a relatively small number of cancer cells may enter the circulatory system, meaning the circulating cancer cells are relatively unlikely to form stable metastases. It follows that early/timely effective treatment of cancer is crucial to relapse prevention and life extension. Currently, the criteria of cancer diagnosis consist in cell and tissue morphology. Morphological observation, however, depends heavily on a pathologist's experience, and missed diagnosis may occur due to the fact that an early-stage cancer may not have developed typical morphological changes. Now that molecular biological changes in epigenetics and genetics take place earlier than variations in morphology during cancer development, molecular biology-based detection is more sensitive than morphology-based detection when it comes to detecting an early-stage cancer. Molecular markers are hence important to early diagnosis of cancer.

Genomic imprinting is a gene regulation method in epigenetics and is characterized by methylating an allele from a specific parent such that only one allele of the corresponding gene is expressed while the other allele is in a silenced state. A gene regulated in this way is referred to as an imprinted gene. Loss of imprinting is an epigenetic change in which the silenced allele of an imprinted gene is demethylated and is thus activated and expressed. Numerous studies have shown that loss of imprinting exists widely in all kinds of cancers and occurs earlier than morphological changes in cells and tissue. Loss of imprinting, however, seldom occurs in healthy cells, which is in stark contrast to the case with cancer cells. Therefore, the methylated state of an imprinted gene can serve as a pathological marker and used in conjunction with specific molecular detection techniques to analyze cellular abnormality.

As the functions of an imprinted gene cover cell signaling, cell cycle regulation, substance transport across the cell membrane, the formation of extracellular matrix, and so on, the expressed functions and expressed quantity of an imprinted gene in a certain cancer may differ from those in another cancer, with the difference in expressed quantity being huge. That is to say, an imprinted gene may have different sensitivity and specificity to a certain cancer from another imprinted gene. Such differences in sensitivity and specificity have a significant impact on the invasion and metastasis of tumor cells during tumor development and on the prognosis of tumors. Different combinations of imprinted genes can also be used to distinguish different types of tumors.

According to the above, highly sensitive diagnostic markers for early tumor diagnosis are currently unavailable. It is necessary to develop a new diagnostic marker, diagnostic model, and diagnostic device that can be applied to a patient's biopsy samples to analyze changes in a molecular marker on a cellular level, in order to provide more accurate prognosis and diagnosis information as well as guidance on the selection of treatment options.

### BRIEF SUMMARY OF THE INVENTION

This application provides a tumor marker and its applications. An imprinted gene is used as a tumor marker for early tumor diagnosis. A tumor diagnosis model and tumor diagnosis device based on the imprinted gene-based tumor marker enable tumor diagnosis on a single-cell or tissue level and thereby provide a basis for the treatment of various malignant tumors.

In its first aspect, this application provides a tumor marker that includes any one, or a combination of at least two, of imprinted genes Z17, Z18, Z19, Z20, Z21, Z22, Z23, Z25, Z26, Z27, and Z28.

The imprinted gene Z17 is Aldh111, the imprinted gene Z18 is Gata3, the imprinted gene Z19 is Hm13, the imprinted gene Z20 is Kcnq1, the imprinted gene Z21 is Nhp211, the imprinted gene Z22 is Pon2, the imprinted gene Z23 is Rasgrf1, the imprinted gene Z25 is Tfpi2, the imprinted gene Z26 is Trappc9, the imprinted gene Z27 is Ube3a, and the imprinted gene Z28 is Zic1.

In this application, the Aldh111 gene has the nucleotide sequence of NC_000003.12 (126103561-126197758, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Gata3 gene has the nucleotide sequence of NC_000010.11 (8045420-8075198) or has a nucleotide sequence showing at least 90%, at least 91%, at least 92%, at least 93%, at least 94%, at least 95%, at least 96%, at least 97%, at least 98%, or at least 99% homology therewith.

In this application, the Hm13 gene has the nucleotide sequence of NC_000020.11 (31514410-31569567) or has a nucleotide sequence showing at least 90%, at least 91%, at least 92%, at least 93%, at least 94%, at least 95%, at least 96%, at least 97%, at least 98%, or at least 99% homology therewith.

In this application, the Kcnq1 gene has the nucleotide sequence of NC_000011.10 (2445008-2849110) or has a nucleotide sequence showing at least 90%, at least 91%, at least 92%, at least 93%, at least 94%, at least 95%, at least 96%, at least 97%, at least 98%, or at least 99% homology therewith.

In this application, the Nhp211 gene has the nucleotide sequence of NC_000022.11 (41673933-41690492, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Pon2 gene has the nucleotide sequence of NC_000007.14 (95404862-95435072, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Rasgrf1 gene has the nucleotide sequence of NC_000015.10 (78959906-79090780, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Tfpi2 gene has the nucleotide sequence of NC_000007.14 (93885396-93890753, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Trappc9 gene has the nucleotide sequence of NC_000008.11 (139727725-140458579, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Ube3a gene has the nucleotide sequence of NC_000015.10 (25333728-25439381, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Zic1 gene has the nucleotide sequence of NC_000003.12 (147409365-147416719) or has a nucleotide sequence showing at least 90%, at least 91%, at least 92%, at least 93%, at least 94%, at least 95%, at least 96%, at least 97%, at least 98%, or at least 99% homology therewith.

In this application, the imprinted genes Z17, Z18, Z19, Z20, Z21, Z22, Z23, Z25, Z26, Z27, and Z28 are not expressed or are expressed as normal in normal tissue or cells; in cancer tissue, however, these imprinted genes are expressed as having a loss of imprinting or a copy number variation in a large number of cells. In this application, one, or a combination of at least two, of the imprinted genes Z17, Z18, Z19, Z20, Z21, Z22, Z23, Z25, Z26, Z27, and Z28 are used as a tumor marker, and highly sensitive tumor diagnosis can be carried out by calculating a total expressed quantity of the imprinted gene(s), an expressed quantity of the imprinted gene(s) being normal, an expressed quantity of the imprinted gene(s) with a loss of imprinting, and an expressed quantity of the imprinted gene(s) with a copy number variation. In particular, Z19 and Z21 show a loss of imprinting or a copy number variation in a large number of cells in the samples of 19 cancers, namely cholangiocarcinoma (bile duct cancer), brain cancer, kidney cancer, bone cancer, cervical cancer, nasopharyngeal carcinoma, laryngeal cancer, prostate cancer, gastric carcinoma, esophageal cancer, mammary cancer, thyroid cancer, bladder cancer, liver cancer, pancreatic cancer, lymphoma, colorectal cancer, skin cancer, and lung cancer; in other words, Z19 and Z21 exhibit significant diagnostic sensitivity to the aforesaid cancers and are therefore highly effective tumor markers.

In some embodiments of this application, the tumor marker further includes any one, or a combination of at least two, of imprinted genes Z1, Z2, Z3, Z4, Z5, Z6, Z8, Z9, Z10, N11, Z12, Z13, Z14, Z15, and N16.

The imprinted gene Z1 is Gnas, the imprinted gene Z2 is Igf2, the imprinted gene Z3 is Peg10, the imprinted gene Z4 is Igf2r, the imprinted gene Z5 is Mest, the imprinted gene Z6 is Plagl1, the imprinted gene Z8 is Dcn, the imprinted gene Z9 is Dlk1, the imprinted gene Z10 is Gatm, the imprinted gene N11 is Grb10, the imprinted gene Z12 is Peg3, the imprinted gene Z13 is Sgce, the imprinted gene Z14 is Slc38a4, the imprinted gene Z15 is Diras3, and the imprinted gene N16 is Snrpn/Snurf.

In this application, the Gnas gene has the nucleotide sequence of NC_000020.11 (58839681-58911196) or has a nucleotide sequence showing at least 90%, at least 91%, at least 92%, at least 93%, at least 94%, at least 95%, at least 96%, at least 97%, at least 98%, or at least 99% homology therewith.

In this application, the Igf2 gene has the nucleotide sequence of NC_000011.10 (2129112-2149603, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Peg10 gene has the nucleotide sequence of NC_000007.14 (94656325-94669695) or has a nucleotide sequence showing at least 90%, at least 91%, at least 92%, at least 93%, at least 94%, at least 95%, at least 96%, at least 97%, at least 98%, or at least 99% homology therewith.

In this application, the Igf2r gene has the nucleotide sequence of NC_000006.12 (159969082-160111504) or has a nucleotide sequence showing at least 90%, at least 91%, at least 92%, at least 93%, at least 94%, at least 95%, at least 96%, at least 97%, at least 98%, or at least 99% homology therewith.

In this application, the Mest gene has the nucleotide sequence of NC_000007.14 (130486175-130506465) or has a nucleotide sequence showing at least 90%, at least 91%, at least 92%, at least 93%, at least 94%, at least 95%, at least 96%, at least 97%, at least 98%, or at least 99% homology therewith.

In this application, the Plagl1 gene has the nucleotide sequence of NC_000006.12 (143940300-144064599, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Dcn gene has the nucleotide sequence of NC_000012.12 (91140484-91182817, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Dlk1 gene has the nucleotide sequence of NC_000014.9 (100726892-100738224) or has a nucleotide sequence showing at least 90%, at least 91%, at least 92%, at least 93%, at least 94%, at least 95%, at least 96%, at least 97%, at least 98%, or at least 99% homology therewith.

In this application, the Gatm gene has the nucleotide sequence of NC_000015.10 (45361124-45402227, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Grb10 gene has the nucleotide sequence of NC_000007.14 (50590068-50793453, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Peg3 gene has the nucleotide sequence of NC_000019.10 (56810082-56840726, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Sgce gene has the nucleotide sequence of NC_000007.14 (94584980-94656205, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Slc38a4 gene has the nucleotide sequence of NC_000012.12 (46764761-46832422, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Diras3 gene has the nucleotide sequence of NC_000001.11 (68045886-68051631, complementary strands) or has a nucleotide sequence showing at least 90%, at least 91%, at least 92%, at least 93%, at least 94%, at least 95%, at least 96%, at least 97%, at least 98%, or at least 99% homology therewith.

In this application, the Snrpn/Snurf gene has the nucleotide sequence of NC_000015.10 (24823637-24978723) or has a nucleotide sequence showing at least 90%, at least 91%, at least 92%, at least 93%, at least 94%, at least 95%, at least 96%, at least 97%, at least 98%, or at least 99% homology therewith.

In this application, the imprinted genes Z1, Z2, Z3, Z4, Z5, Z6, Z8, Z9, Z10, N11, Z12, Z13, Z14, Z15, and N16 are all highly sensitive tumor markers. They can be used to identify various tumor samples accurately because they are not expressed or are expressed as normal in normal tissue or cells but show a loss of imprinting or a copy number variation in a large number of cells in cancer tissue.

Preferably, the aforesaid tumor includes any one, or a combination of at least two, of cholangiocarcinoma, brain cancer, kidney cancer, bone cancer, cervical cancer, nasopharyngeal carcinoma, and laryngeal cancer.

Preferably, the aforesaid tumor further includes any one, or a combination of at least two, of prostate cancer, gastric carcinoma, esophageal cancer, mammary cancer, thyroid cancer, bladder cancer, liver cancer, pancreatic cancer, lymphoma, colorectal cancer, skin cancer, and lung cancer.

According to this application, the combinations of imprinted genes that show a loss of imprinting or a copy number variation in cancer samples vary from one cancer to another. The imprinted gene combinations that can be used as tumor markers for the aforesaid 19 cancers are as follows.

In one embodiment, the tumor marker is a marker for cholangiocarcinoma and includes any one, or a combination of at least two, of the imprinted genes Z1, Z2, Z8, Z10, N11, Z14, N16, Z19, Z20, Z21, Z22, Z25, Z26, and Z27.

In one embodiment, the tumor marker is a marker for brain cancer and includes any one, or a combination of at least two, of the imprinted genes Z1, Z2, Z4, Z5, Z8, Z9, Z10, N11, Z13, Z14, N16, Z17, Z19, Z21, Z22, Z23, Z26, Z27, and Z28.

In one embodiment, the tumor marker is a marker for kidney cancer and includes any one, or a combination of at least two, of the imprinted genes Z1, Z4, Z6, Z8, Z10, N11, Z13, N16, Z17, Z18, Z19, Z20, Z21, Z25, Z26, Z27, and Z28.

In one embodiment, the tumor marker is a marker for bone cancer and includes any one, or a combination of at least two, of the imprinted genes Z1, Z2, Z3, Z6, Z8, Z9, Z10, N11, Z13, N16, Z18, Z19, Z20, Z21, Z22, Z25, Z26, Z27, and Z28.

In one embodiment, the tumor marker is a marker for cervical cancer and includes any one, or a combination of at least two, of the imprinted genes Z1, Z3, Z5, Z8, Z9, Z10, N11, N16, Z17, Z19, Z20, Z21, Z22, Z23, Z26, Z27, and Z28.

In one embodiment, the tumor marker is a marker for nasopharyngeal carcinoma and includes any one, or a combination of at least two, of the imprinted genes Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, and Z28.

In one embodiment, the tumor marker is a marker for laryngeal cancer and includes any one, or a combination of at least two, of the imprinted genes Z1, Z3, Z6, Z8, N11, Z15, N16, Z17, Z18, Z19, Z21, Z22, Z25, Z26, Z27, and Z28.

In one embodiment, the tumor marker is a marker for prostate cancer and includes any one, or a combination of at least two, of the imprinted genes Z18, Z19, Z20, Z21, Z25, Z26, Z27, and Z28.

In one embodiment, the tumor marker is a marker for gastric carcinoma and includes any one, or a combination of at least two, of the imprinted genes Z18, Z19, Z20, Z21, Z22, Z26, and Z27.

In one embodiment, the tumor marker is a marker for esophageal cancer and includes any one, or a combination of at least two, of the imprinted genes Z18, Z19, Z21, Z22, Z26, Z27, and Z28.

In one embodiment, the tumor marker is a marker for mammary cancer and includes any one, or a combination of at least two, of the imprinted genes Z18, Z19, Z21, Z22, and Z27.

In one embodiment, the tumor marker is a marker for thyroid cancer and includes any one, or a combination of at least two, of the imprinted genes Z18, Z19, Z20, Z21, and Z23.

In one embodiment, the tumor marker is a marker for bladder cancer and includes any one, or a combination of at least two, of the imprinted genes Z18, Z19, Z20, Z21, Z22, Z26, Z27, and Z28.

In one embodiment, the tumor marker is a marker for liver cancer and includes any one, or a combination of at least two, of the imprinted genes Z17, Z19, Z21, Z22, Z26, Z27, and Z28.

In one embodiment, the tumor marker is a marker for pancreatic cancer and includes any one, or a combination of at least two, of the imprinted genes Z17, Z18, Z19, Z20, Z21, Z22, Z26, Z27, and Z28.

In one embodiment, the tumor marker is a marker for lymphoma and includes any one, or a combination of at least two, of the imprinted genes Z18, Z19, Z21, Z22, Z26, Z27, and Z28.

In one embodiment, the tumor marker is a marker for colorectal cancer and includes any one, or a combination of at least two, of the imprinted genes Z19, Z20, Z21, Z22, Z26, and Z27.

In one embodiment, the tumor marker is a marker for skin cancer and includes any one, or a combination of at least two, of the imprinted genes Z18, Z19, Z21, Z22, Z25, and Z28.

In one embodiment, the tumor marker is a marker for lung cancer and includes any one, or a combination of at least two, of the imprinted genes Z18, Z19, Z21, Z22, Z25, Z26, Z27, and Z28.

In its second aspect, this application provides a tumor diagnosis model that carries out tumor diagnosis by analyzing how the aforesaid tumor markers (i.e., imprinted genes) are expressed in samples.

Expression of the aforesaid imprinted genes includes any one, or a combination of at least two, of an imprinted gene not being expressed, the imprinted gene being expressed as normal, the imprinted gene being expressed as having a loss of imprinting, and the imprinted gene being expressed as having a copy number variation.

In this application, an imprinted gene is determined as having a loss of imprinting when two red/brown marks are present in the nucleus of a cell stained with hematoxylin, and an imprinted gene is determined as having a copy number variation when more than two red/brown marks are present in the nucleus of a cell stained with hematoxylin. A copy number variation occurs when abnormal gene duplication takes place in a cancer cell such that the duplicated gene is expressed as having three or more copies of the original gene.

In more general terms, an imprinted gene is determined as not being expressed when no imprinted gene mark is present in the nucleus of a sample cell.

In more general terms, an imprinted gene is determined as being expressed as normal when only one imprinted gene mark is present in the nucleus of a sample cell.

In more general terms, an imprinted gene is determined as having a loss of imprinting when two imprinted gene marks are present in the nucleus of a sample cell.

In more general terms, an imprinted gene is determined as having a copy number variation when more than two imprinted gene marks are present in the nucleus of a sample cell.

In this application, the hematoxylin-stained marks include but are not limited to red marks and brown marks. Staining/marking in other colors is also feasible when analyzing imprinted gene expression.

According to this application, a probe/probes specific to the imprinted gene(s) to be detected in a sample is/are used for in-situ hybridization with the sample, and tumor diagnosis is carried out by analyzing whether the imprinted gene(s) in the sample cells has/have a loss of imprinting or a copy number variation.

The diagnostic method of the tumor diagnosis model is detailed as follows.

In one embodiment of this application, a bile duct tissue and/or cell sample is subjected to an analysis of the expression of any one, or a combination of at least two, of the imprinted genes Z1, Z2, Z8, Z10, N11, Z14, N16, Z19, Z20, Z21, Z22, Z25, Z26, and Z27.

If the imprinted gene Z1, Z2, Z8, Z10, N11, Z14, N16, Z19, Z20, Z21, Z22, Z25, Z26, or Z27 is not expressed or is expressed as normal in the cell nuclei in the bile duct tissue and/or cell sample, the sample will be determined as a healthy bile duct tissue and/or cell sample.

If, however, the imprinted gene Z1, Z2, Z8, Z10, N11, Z14, N16, Z19, Z20, Z21, Z22, Z25, Z26, or Z27 is expressed as having a loss of imprinting and/or a copy number variation in the cell nuclei in the bile duct tissue and/or cell sample, the sample will be determined as a cholangiocarcinoma tissue and/or cell sample.

In one embodiment of this application, a brain tissue and/or cell sample is subjected to an analysis of the expression of any one, or a combination of at least two, of the imprinted genes Z1, Z2, Z4, Z5, Z8, Z9, Z10, N11, Z13, Z14, N16, Z17, Z19, Z21, Z22, Z23, Z26, Z27, and Z28.

If the imprinted gene Z1, Z2, Z4, Z5, Z8, Z9, Z10, N11, Z13, Z14, N16, Z17, Z19, Z21, Z22, Z23, Z26, Z27, or Z28 is not expressed or is expressed as normal in the cell nuclei in the brain tissue and/or cell sample, the sample will be determined as a healthy brain tissue and/or cell sample.

If, however, the imprinted gene Z1, Z2, Z4, Z5, Z8, Z9, Z10, N11, Z13, Z14, N16, Z17, Z19, Z21, Z22, Z23, Z26, Z27, or Z28 is expressed as having a loss of imprinting and/or a copy number variation in the cell nuclei in the brain tissue and/or cell sample, the sample will be determined as a brain cancer tissue and/or cell sample.

In one embodiment of this application, a kidney tissue and/or cell sample is subjected to an analysis of the expression of any one, or a combination of at least two, of the imprinted genes Z1, Z4, Z6, Z8, Z10, N11, Z13, N16, Z17, Z18, Z19, Z20, Z21, Z25, Z26, Z27, and Z28.

If the imprinted gene Z1, Z4, Z6, Z8, Z10, N11, Z13, N16, Z17, Z18, Z19, Z20, Z21, Z25, Z26, Z27, or Z28 is not expressed or is expressed as normal in the cell nuclei in the kidney tissue and/or cell sample, the sample will be determined as a healthy kidney tissue and/or cell sample.

If, however, the imprinted gene Z1, Z4, Z6, Z8, Z10, N11, Z13, N16, Z17, Z18, Z19, Z20, Z21, Z25, Z26, Z27, or Z28 is expressed as having a loss of imprinting and/or a copy number variation in the cell nuclei in the kidney tissue and/or cell sample, the sample will be determined as a kidney cancer tissue and/or cell sample.

In one embodiment of this application, a bone tissue and/or cell sample is subjected to an analysis of the expression of any one, or a combination of at least two, of the imprinted genes Z1, Z2, Z3, Z6, Z8, Z9, Z10, N11, Z13, N16, Z18, Z19, Z20, Z21, Z22, Z25, Z26, Z27, and Z28.

If the imprinted gene Z1, Z2, Z3, Z6, Z8, Z9, Z10, N11, Z13, N16, Z18, Z19, Z20, Z21, Z22, Z25, Z26, Z27, or Z28 is not expressed or is expressed as normal in the cell nuclei in the bone tissue and/or cell sample, the sample will be determined as a healthy bone tissue and/or cell sample.

If, however, the imprinted gene Z1, Z2, Z3, Z6, Z8, Z9, Z10, N11, Z13, N16, Z18, Z19, Z20, Z21, Z22, Z25, Z26, Z27, or Z28 is expressed as having a loss of imprinting and/or a copy number variation in the cell nuclei in the bone tissue and/or cell sample, the sample will be determined as a bone cancer tissue and/or cell sample.

In one embodiment of this application, a cervical tissue and/or cell sample is subjected to an analysis of the expression of any one, or a combination of at least two, of the imprinted genes Z1, Z3, Z5, Z8, Z9, Z10, N11, N16, Z17, Z19, Z20, Z21, Z22, Z23, Z26, Z27, and Z28.

If the imprinted gene Z1, Z3, Z5, Z8, Z9, Z10, N11, N16, Z17, Z19, Z20, Z21, Z22, Z23, Z26, Z27, or Z28 is not expressed or is expressed as normal in the cell nuclei in the cervical tissue and/or cell sample, the sample will be determined as a healthy cervical tissue and/or cell sample.

If, however, the imprinted gene Z1, Z3, Z5, Z8, Z9, Z10, N11, N16, Z17, Z19, Z20, Z21, Z22, Z23, Z26, Z27, or Z28 is expressed as having a loss of imprinting and/or a copy number variation in the cell nuclei in the cervical tissue and/or cell sample, the sample will be determined as a cervical cancer tissue and/or cell sample.

In one embodiment of this application, a nasopharyngeal tissue and/or cell sample is subjected to an analysis of the expression of any one, or a combination of at least two, of the imprinted genes Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, and Z28.

If the imprinted gene Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, or Z28 is not expressed or is expressed as normal in the cell nuclei in the nasopharyngeal tissue and/or cell sample, the sample will be determined as a healthy nasopharyngeal tissue and/or cell sample.

If, however, the imprinted gene Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, or Z28 is expressed as having a loss of imprinting and/or a copy number variation in the cell nuclei in the nasopharyngeal tissue and/or cell sample, the sample will be determined as a nasopharyngeal carcinoma tissue and/or cell sample.

In one embodiment of this application, a laryngeal tissue and/or cell sample is subjected to an analysis of the expression of any one, or a combination of at least two, of the imprinted genes Z1, Z3, Z6, Z8, N11, Z15, N16, Z17, Z18, Z19, Z21, Z22, Z25, Z26, Z27, and Z28.

If the imprinted gene Z1, Z3, Z6, Z8, N11, Z15, N16, Z17, Z18, Z19, Z21, Z22, Z25, Z26, Z27, or Z28 is not expressed or is expressed as normal in the cell nuclei in the laryngeal tissue and/or cell sample, the sample will be determined as a healthy laryngeal tissue and/or cell sample.

If, however, the imprinted gene Z1, Z3, Z6, Z8, N11, Z15, N16, Z17, Z18, Z19, Z21, Z22, Z25, Z26, Z27, or Z28 is expressed as having a loss of imprinting and/or a copy number variation in the cell nuclei in the laryngeal tissue and/or cell sample, the sample will be determined as a laryngeal cancer tissue and/or cell sample.

In one embodiment of this application, a prostate tissue and/or cell sample is subjected to an analysis of the expression of any one, or a combination of at least two, of the imprinted genes Z18, Z19, Z20, Z21, Z25, Z26, Z27, and Z28.

If the imprinted gene Z18, Z19, Z20, Z21, Z25, Z26, Z27, or Z28 is not expressed or is expressed as normal in the cell nuclei in the prostate tissue and/or cell sample, the sample will be determined as a healthy prostate tissue and/or cell sample.

If, however, the imprinted gene Z18, Z19, Z20, Z21, Z25, Z26, Z27, or Z28 is expressed as having a loss of imprinting and/or a copy number variation in the cell nuclei in the prostate tissue and/or cell sample, the sample will be determined as a prostate cancer tissue and/or cell sample.

In one embodiment of this application, a gastric tissue and/or cell sample is subjected to an analysis of the expression of any one, or a combination of at least two, of the imprinted genes Z18, Z19, Z20, Z21, Z22, Z26, and Z27.

If the imprinted gene Z18, Z19, Z20, Z21, Z22, Z26, or Z27 is not expressed or is expressed as normal in the cell nuclei in the gastric tissue and/or cell sample, the sample will be determined as a healthy gastric tissue and/or cell sample.

If, however, the imprinted gene Z18, Z19, Z20, Z21, Z22, Z26, or Z27 is expressed as having a loss of imprinting and/or a copy number variation in the cell nuclei in the gastric tissue and/or cell sample, the sample will be determined as a gastric carcinoma tissue and/or cell sample.

In one embodiment of this application, an esophageal tissue and/or cell sample is subjected to an analysis of the expression of any one, or a combination of at least two, of the imprinted genes Z18, Z19, Z21, Z22, Z26, Z27, and Z28.

If the imprinted gene Z18, Z19, Z21, Z22, Z26, Z27, or Z28 is not expressed or is expressed as normal in the cell nuclei in the esophageal tissue and/or cell sample, the sample will be determined as a healthy esophageal tissue and/or cell sample.

If, however, the imprinted gene Z18, Z19, Z21, Z22, Z26, Z27, or Z28 is expressed as having a loss of imprinting and/or a copy number variation in the cell nuclei in the esophageal tissue and/or cell sample, the sample will be determined as an esophageal cancer tissue and/or cell sample.

In one embodiment of this application, a mammary tissue and/or cell sample is subjected to an analysis of the expression of any one, or a combination of at least two, of the imprinted genes Z18, Z19, Z21, Z22, and Z27.

If the imprinted gene Z18, Z19, Z21, Z22, or Z27 is not expressed or is expressed as normal in the cell nuclei in the mammary tissue and/or cell sample, the sample will be determined as a healthy mammary tissue and/or cell sample.

If, however, the imprinted gene Z18, Z19, Z21, Z22, or Z27 is expressed as having a loss of imprinting and/or a copy number variation in the cell nuclei in the mammary tissue and/or cell sample, the sample will be determined as a mammary cancer tissue and/or cell sample.

In one embodiment of this application, a thyroid tissue and/or cell sample is subjected to an analysis of the expression of any one, or a combination of at least two, of the imprinted genes Z18, Z19, Z20, Z21, and Z23.

If the imprinted gene Z18, Z19, Z20, Z21, or Z23 is not expressed or is expressed as normal in the cell nuclei in the thyroid tissue and/or cell sample, the sample will be determined as a healthy thyroid tissue and/or cell sample.

If, however, the imprinted gene Z18, Z19, Z20, Z21, or Z23 is expressed as having a loss of imprinting and/or a copy number variation in the cell nuclei in the thyroid tissue and/or cell sample, the sample will be determined as a thyroid cancer tissue and/or cell sample.

In one embodiment of this application, a bladder tissue and/or cell sample is subjected to an analysis of the expression of any one, or a combination of at least two, of the imprinted genes Z18, Z19, Z20, Z21, Z22, Z26, Z27, and Z28.

If the imprinted gene Z18, Z19, Z20, Z21, Z22, Z26, Z27, or Z28 is not expressed or is expressed as normal in the cell nuclei in the bladder tissue and/or cell sample, the sample will be determined as a healthy bladder tissue and/or cell sample.

If, however, the imprinted gene Z18, Z19, Z20, Z21, Z22, Z26, Z27, or Z28 is expressed as having a loss of imprinting and/or a copy number variation in the cell nuclei in the bladder tissue and/or cell sample, the sample will be determined as a bladder cancer tissue and/or cell sample.

In one embodiment of this application, a liver tissue and/or cell sample is subjected to an analysis of the expression of any one, or a combination of at least two, of the imprinted genes Z17, Z19, Z21, Z22, Z26, Z27, and Z28.

If the imprinted gene Z17, Z19, Z21, Z22, Z26, Z27, or Z28 is not expressed or is expressed as normal in the cell nuclei in the liver tissue and/or cell sample, the sample will be determined as a healthy liver tissue and/or cell sample.

If, however, the imprinted gene Z17, Z19, Z21, Z22, Z26, Z27, or Z28 is expressed as having a loss of imprinting and/or a copy number variation in the cell nuclei in the liver tissue and/or cell sample, the sample will be determined as a liver cancer tissue and/or cell sample.

In one embodiment of this application, a pancreatic tissue and/or cell sample is subjected to an analysis of the expression of any one, or a combination of at least two, of the imprinted genes Z17, Z18, Z19, Z20, Z21, Z22, Z26, Z27, and Z28.

If the imprinted gene Z17, Z18, Z19, Z20, Z21, Z22, Z26, Z27, or Z28 is not expressed or is expressed as normal in the cell nuclei in the pancreatic tissue and/or cell sample, the sample will be determined as a healthy pancreatic tissue and/or cell sample.

If, however, the imprinted gene Z17, Z18, Z19, Z20, Z21, Z22, Z26, Z27, or Z28 is expressed as having a loss of imprinting and/or a copy number variation in the cell nuclei in the pancreatic tissue and/or cell sample, the sample will be determined as a pancreatic cancer tissue and/or cell sample.

In one embodiment of this application, a lymphatic tissue and/or cell sample is subjected to an analysis of the expression of any one, or a combination of at least two, of the imprinted genes Z18, Z19, Z21, Z22, Z26, Z27, and Z28.

If the imprinted gene Z18, Z19, Z21, Z22, Z26, Z27, or Z28 is not expressed or is expressed as normal in the cell nuclei in the lymphatic tissue and/or cell sample, the sample will be determined as a healthy lymphatic tissue and/or cell sample.

If, however, the imprinted gene Z18, Z19, Z21, Z22, Z26, Z27, or Z28 is expressed as having a loss of imprinting and/or a copy number variation in the cell nuclei in the lymphatic tissue and/or cell sample, the sample will be determined as a lymphoma tissue and/or cell sample.

In one embodiment of this application, a colorectal tissue and/or cell sample is subjected to an analysis of the expression of any one, or a combination of at least two, of the imprinted genes Z19, Z20, Z21, Z22, Z26, and Z27.

If the imprinted gene Z19, Z20, Z21, Z22, Z26, or Z27 is not expressed or is expressed as normal in the cell nuclei in the colorectal tissue and/or cell sample, the sample will be determined as a healthy colorectal tissue and/or cell sample.

If, however, the imprinted gene Z19, Z20, Z21, Z22, Z26, or Z27 is expressed as having a loss of imprinting and/or a copy number variation in the cell nuclei in the colorectal tissue and/or cell sample, the sample will be determined as a colorectal cancer tissue and/or cell sample.

In one embodiment of this application, a skin tissue and/or cell sample is subjected to an analysis of the expression of any one, or a combination of at least two, of the imprinted genes Z18, Z19, Z21, Z22, Z25, and Z28.

If the imprinted gene Z18, Z19, Z21, Z22, Z25, or Z28 is not expressed or is expressed as normal in the cell nuclei in the skin tissue and/or cell sample, the sample will be determined as a healthy skin tissue and/or cell sample.

If, however, the imprinted gene Z18, Z19, Z21, Z22, Z25, or Z28 is expressed as having a loss of imprinting and/or a copy number variation in the cell nuclei in the skin tissue and/or cell sample, the sample will be determined as a skin cancer tissue and/or cell sample.

In one embodiment of this application, a lung tissue and/or cell sample is subjected to an analysis of the expression of any one, or a combination of at least two, of the imprinted genes Z18, Z19, Z21, Z22, Z25, Z26, Z27, and Z28.

If the imprinted gene Z18, Z19, Z21, Z22, Z25, Z26, Z27, or Z28 is not expressed or is expressed as normal in the cell nuclei in the lung tissue and/or cell sample, the sample will be determined as a healthy lung tissue and/or cell sample.

If, however, the imprinted gene Z18, Z19, Z21, Z22, Z25, Z26, Z27, or Z28 is expressed as having a loss of imprinting and/or a copy number variation in the cell nuclei in the lung tissue and/or cell sample, the sample will be determined as a lung cancer tissue and/or cell sample.

In its third aspect, this application provides a tumor diagnosis device that includes the following units:

a sampling unit for obtaining a sample;

a probe designing unit for designing a probe specific to the sequence of an imprinted gene to be detected;

a detection unit for performing in-situ hybridization between the probe and the sample; and

an analysis unit for analyzing expression of the imprinted gene through microscopic imaging;

wherein the analysis unit carries out tumor diagnosis by analyzing how the imprinted gene(s) of a tumor marker stated in relation to the first aspect is/are expressed in the sample.

In this application, an imprinted gene is determined as having a loss of imprinting when two red/brown marks are present in the nucleus of a cell stained with hematoxylin, and an imprinted gene is determined as having a copy number variation when more than two red/brown marks are present in the nucleus of a cell stained with hematoxylin. A copy number variation occurs when abnormal gene duplication takes place in a cancer cell such that the duplicated gene is expressed as having three or more copies of the original gene.

In this application, the hematoxylin-stained marks include but are not limited to red marks and brown marks. Staining/marking in other colors is also feasible when analyzing imprinted gene expression.

According to this application, a probe/probes specific to the imprinted gene(s) to be detected in a sample is/are used for in-situ hybridization with the sample, and tumor diagnosis is carried out by analyzing whether the imprinted gene(s) in the sample cells has/have a loss of imprinting or a copy number variation.

In one embodiment of this application, the sample is sourced from tissue and/or cells.

In this application, it is required that samples be fixed with formalin to prevent RNA degradation. The tissue samples for use may be paraffin sections, and the steps of obtaining a paraffin section include obtaining a human tumor tissue sample, fixing the tissue with 10% neutral buffered formalin in time, embedding the tissue in paraffin, cutting a 10-μm section out of the paraffin-embedded tissue, and making a tissue slide with a positively charged glass slide.

In one embodiment of this application, the in-situ hybridization uses an RNAscope in-situ hybridization method.

In this application, the probes for use in the in-situ hybridization are designed according to the sequences of the imprinted genes to be detected. More specifically, a sequence is selected from the intron of each of the to-be-detected genes as the corresponding probe. The probes used in this application were designed by Advanced Cell Diagnostics.

In one embodiment of this application, the RNAscope in-situ hybridization method uses a single- or multiple-channel chromogenic reagent kit or a single- or multiple-channel fluorescent reagent kit, preferably a single-channel red/brown chromogenic reagent kit or a multiple-channel fluorescent reagent kit.

The analysis method of the tumor diagnosis device is detailed as follows.

In one embodiment of this application, a bile duct tissue and/or cell sample is subjected to an analysis of the expression of any one, or a combination of at least two, of the imprinted genes Z1, Z2, Z8, Z10, N11, Z14, N16, Z19, Z20, Z21, Z22, Z25, Z26, and Z27.

If the imprinted gene Z1, Z2, Z8, Z10, N11, Z14, N16, Z19, Z20, Z21, Z22, Z25, Z26, or Z27 is not expressed or is expressed as normal in the cell nuclei in the bile duct tissue and/or cell sample, the sample will be determined as a healthy bile duct tissue and/or cell sample.

If, however, the imprinted gene Z1, Z2, Z8, Z10, N11, Z14, N16, Z19, Z20, Z21, Z22, Z25, Z26, or Z27 is expressed as having a loss of imprinting and/or a copy number variation in the cell nuclei in the bile duct tissue and/or cell sample, the sample will be determined as a cholangiocarcinoma tissue and/or cell sample.

In one embodiment of this application, a brain tissue and/or cell sample is subjected to an analysis of the expression of any one, or a combination of at least two, of the imprinted genes Z1, Z2, Z4, Z5, Z8, Z9, Z10, N11, Z13, Z14, N16, Z17, Z19, Z21, Z22, Z23, Z26, Z27, and Z28.

If the imprinted gene Z1, Z2, Z4, Z5, Z8, Z9, Z10, N11, Z13, Z14, N16, Z17, Z19, Z21, Z22, Z23, Z26, Z27, or Z28 is not expressed or is expressed as normal in the cell nuclei in the brain tissue and/or cell sample, the sample will be determined as a healthy brain tissue and/or cell sample.

If, however, the imprinted gene Z1, Z2, Z4, Z5, Z8, Z9, Z10, N11, Z13, Z14, N16, Z17, Z19, Z21, Z22, Z23, Z26, Z27, or Z28 is expressed as having a loss of imprinting and/or a copy number variation in the cell nuclei in the brain tissue and/or cell sample, the sample will be determined as a brain cancer tissue and/or cell sample.

In one embodiment of this application, a kidney tissue and/or cell sample is subjected to an analysis of the expression of any one, or a combination of at least two, of the imprinted genes Z1, Z4, Z6, Z8, Z10, N11, Z13, N16, Z17, Z18, Z19, Z20, Z21, Z25, Z26, Z27, and Z28.

If the imprinted gene Z1, Z4, Z6, Z8, Z10, N11, Z13, N16, Z17, Z18, Z19, Z20, Z21, Z25, Z26, Z27, or Z28 is not expressed or is expressed as normal in the cell nuclei in the kidney tissue and/or cell sample, the sample will be determined as a healthy kidney tissue and/or cell sample.

If, however, the imprinted gene Z1, Z4, Z6, Z8, Z10, N11, Z13, N16, Z17, Z18, Z19, Z20, Z21, Z25, Z26, Z27, or Z28 is expressed as having a loss of imprinting and/or a copy number variation in the cell nuclei in the kidney tissue and/or cell sample, the sample will be determined as a kidney cancer tissue and/or cell sample.

In one embodiment of this application, a bone tissue and/or cell sample is subjected to an analysis of the expression of any one, or a combination of at least two, of the imprinted genes Z1, Z2, Z3, Z6, Z8, Z9, Z10, N11, Z13, N16, Z18, Z19, Z20, Z21, Z22, Z25, Z26, Z27, and Z28.

If the imprinted gene Z1, Z2, Z3, Z6, Z8, Z9, Z10, N11, Z13, N16, Z18, Z19, Z20, Z21, Z22, Z25, Z26, Z27, or Z28 is not expressed or is expressed as normal in the cell nuclei in the bone tissue and/or cell sample, the sample will be determined as a healthy bone tissue and/or cell sample.

If, however, the imprinted gene Z1, Z2, Z3, Z6, Z8, Z9, Z10, N11, Z13, N16, Z18, Z19, Z20, Z21, Z22, Z25, Z26, Z27, or Z28 is expressed as having a loss of imprinting and/or a copy number variation in the cell nuclei in the bone tissue and/or cell sample, the sample will be determined as a bone cancer tissue and/or cell sample.

In one embodiment of this application, a cervical tissue and/or cell sample is subjected to an analysis of the expression of any one, or a combination of at least two, of the imprinted genes Z1, Z3, Z5, Z8, Z9, Z10, N11, N16, Z17, Z19, Z20, Z21, Z22, Z23, Z26, Z27, and Z28.

If the imprinted gene Z1, Z3, Z5, Z8, Z9, Z10, N11, N16, Z17, Z19, Z20, Z21, Z22, Z23, Z26, Z27, or Z28 is not expressed or is expressed as normal in the cell nuclei in the cervical tissue and/or cell sample, the sample will be determined as a healthy cervical tissue and/or cell sample.

If, however, the imprinted gene Z1, Z3, Z5, Z8, Z9, Z10, N11, N16, Z17, Z19, Z20, Z21, Z22, Z23, Z26, Z27, or Z28 is expressed as having a loss of imprinting and/or a copy number variation in the cell nuclei in the cervical tissue and/or cell sample, the sample will be determined as a cervical cancer tissue and/or cell sample.

In one embodiment of this application, a nasopharyngeal tissue and/or cell sample is subjected to an analysis of the expression of any one, or a combination of at least two, of the imprinted genes Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, and Z28.

If the imprinted gene Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, or Z28 is not expressed or is expressed as normal in the cell nuclei in the nasopharyngeal tissue and/or cell sample, the sample will be determined as a healthy nasopharyngeal tissue and/or cell sample.

If, however, the imprinted gene Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, or Z28 is expressed as having a loss of imprinting and/or a copy number variation in the cell nuclei in the nasopharyngeal tissue and/or cell sample, the sample will be determined as a nasopharyngeal carcinoma tissue and/or cell sample.

In one embodiment of this application, a laryngeal tissue and/or cell sample is subjected to an analysis of the expression of any one, or a combination of at least two, of the imprinted genes Z1, Z3, Z6, Z8, N11, Z15, N16, Z17, Z18, Z19, Z21, Z22, Z25, Z26, Z27, and Z28.

If the imprinted gene Z1, Z3, Z6, Z8, N11, Z15, N16, Z17, Z18, Z19, Z21, Z22, Z25, Z26, Z27, or Z28 is not expressed or is expressed as normal in the cell nuclei in the laryngeal tissue and/or cell sample, the sample will be determined as a healthy laryngeal tissue and/or cell sample.

If, however, the imprinted gene Z1, Z3, Z6, Z8, N11, Z15, N16, Z17, Z18, Z19, Z21, Z22, Z25, Z26, Z27, or Z28 is expressed as having a loss of imprinting and/or a copy number variation in the cell nuclei in the laryngeal tissue and/or cell sample, the sample will be determined as a laryngeal cancer tissue and/or cell sample.

In one embodiment of this application, a prostate tissue and/or cell sample is subjected to an analysis of the expression of any one, or a combination of at least two, of the imprinted genes Z18, Z19, Z20, Z21, Z25, Z26, Z27, and Z28.

If the imprinted gene Z18, Z19, Z20, Z21, Z25, Z26, Z27, or Z28 is not expressed or is expressed as normal in the cell nuclei in the prostate tissue and/or cell sample, the sample will be determined as a healthy prostate tissue and/or cell sample.

If, however, the imprinted gene Z18, Z19, Z20, Z21, Z25, Z26, Z27, or Z28 is expressed as having a loss of imprinting and/or a copy number variation in the cell nuclei in the prostate tissue and/or cell sample, the sample will be determined as a prostate cancer tissue and/or cell sample.

In one embodiment of this application, a gastric tissue and/or cell sample is subjected to an analysis of the expression of any one, or a combination of at least two, of the imprinted genes Z18, Z19, Z20, Z21, Z22, Z26, and Z27.

If the imprinted gene Z18, Z19, Z20, Z21, Z22, Z26, or Z27 is not expressed or is expressed as normal in the cell nuclei in the gastric tissue and/or cell sample, the sample will be determined as a healthy gastric tissue and/or cell sample.

If, however, the imprinted gene Z18, Z19, Z20, Z21, Z22, Z26, or Z27 is expressed as having a loss of imprinting and/or a copy number variation in the cell nuclei in the gastric tissue and/or cell sample, the sample will be determined as a gastric carcinoma tissue and/or cell sample.

In one embodiment of this application, an esophageal tissue and/or cell sample is subjected to an analysis of the expression of any one, or a combination of at least two, of the imprinted genes Z18, Z19, Z21, Z22, Z26, Z27, and Z28.

If the imprinted gene Z18, Z19, Z21, Z22, Z26, Z27, or Z28 is not expressed or is expressed as normal in the cell nuclei in the esophageal tissue and/or cell sample, the sample will be determined as a healthy esophageal tissue and/or cell sample.

If, however, the imprinted gene Z18, Z19, Z21, Z22, Z26, Z27, or Z28 is expressed as having a loss of imprinting and/or a copy number variation in the cell nuclei in the esophageal tissue and/or cell sample, the sample will be determined as an esophageal cancer tissue and/or cell sample.

In one embodiment of this application, a mammary tissue and/or cell sample is subjected to an analysis of the expression of any one, or a combination of at least two, of the imprinted genes Z18, Z19, Z21, Z22, and Z27.

If the imprinted gene Z18, Z19, Z21, Z22, or Z27 is not expressed or is expressed as normal in the cell nuclei in the mammary tissue and/or cell sample, the sample will be determined as a healthy mammary tissue and/or cell sample.

If, however, the imprinted gene Z18, Z19, Z21, Z22, or Z27 is expressed as having a loss of imprinting and/or a copy number variation in the cell nuclei in the mammary tissue and/or cell sample, the sample will be determined as a mammary cancer tissue and/or cell sample.

In one embodiment of this application, a thyroid tissue and/or cell sample is subjected to an analysis of the expression of any one, or a combination of at least two, of the imprinted genes Z18, Z19, Z20, Z21, and Z23.

If the imprinted gene Z18, Z19, Z20, Z21, or Z2 is not expressed or is expressed as normal in the cell nuclei in the thyroid tissue and/or cell sample, the sample will be determined as a healthy thyroid tissue and/or cell sample.

If, however, the imprinted gene Z18, Z19, Z20, Z21, or Z23 is expressed as having a loss of imprinting and/or a copy number variation in the cell nuclei in the thyroid tissue and/or cell sample, the sample will be determined as a thyroid cancer tissue and/or cell sample.

In one embodiment of this application, a bladder tissue and/or cell sample is subjected to an analysis of the expression of any one, or a combination of at least two, of the imprinted genes Z18, Z19, Z20, Z21, Z22, Z26, Z27, and Z28.

If the imprinted gene Z18, Z19, Z20, Z21, Z22, Z26, Z27, or Z28 is not expressed or is expressed as normal in the cell nuclei in the bladder tissue and/or cell sample, the sample will be determined as a healthy bladder tissue and/or cell sample.

If, however, the imprinted gene Z18, Z19, Z20, Z21, Z22, Z26, Z27, or Z2 is expressed as having a loss of imprinting and/or a copy number variation in the cell nuclei in the bladder tissue and/or cell sample, the sample will be determined as a bladder cancer tissue and/or cell sample.

In one embodiment of this application, a liver tissue and/or cell sample is subjected to an analysis of the expression of any one, or a combination of at least two, of the imprinted genes Z17, Z19, Z21, Z22, Z26, Z27, and Z28.

If the imprinted gene Z17, Z19, Z21, Z22, Z26, Z27, or Z28 is not expressed or is expressed as normal in the cell nuclei in the liver tissue and/or cell sample, the sample will be determined as a healthy liver tissue and/or cell sample.

If, however, the imprinted gene Z17, Z19, Z21, Z22, Z26, Z27, or Z28 is expressed as having a loss of imprinting and/or a copy number variation in the cell nuclei in the liver tissue and/or cell sample, the sample will be determined as a liver cancer tissue and/or cell sample.

In one embodiment of this application, a pancreatic tissue and/or cell sample is subjected to an analysis of the expression of any one, or a combination of at least two, of the imprinted genes Z17, Z18, Z19, Z20, Z21, Z22, Z26, Z27, and Z28.

If the imprinted gene Z17, Z18, Z19, Z20, Z21, Z22, Z26, Z27, or Z28 is not expressed or is expressed as normal in the cell nuclei in the pancreatic tissue and/or cell sample, the sample will be determined as a healthy pancreatic tissue and/or cell sample.

If, however, the imprinted gene Z17, Z18, Z19, Z20, Z21, Z22, Z26, Z27, or Z28 is expressed as having a loss of imprinting and/or a copy number variation in the cell nuclei in the pancreatic tissue and/or cell sample, the sample will be determined as a pancreatic cancer tissue and/or cell sample.

In one embodiment of this application, a lymphatic tissue and/or cell sample is subjected to an analysis of the expression of any one, or a combination of at least two, of the imprinted genes Z18, Z19, Z21, Z22, Z26, Z27, and Z28.

If the imprinted gene Z18, Z19, Z21, Z22, Z26, Z27, or Z28 is not expressed or is expressed as normal in the cell nuclei in the lymphatic tissue and/or cell sample, the sample will be determined as a healthy lymphatic tissue and/or cell sample.

If, however, the imprinted gene Z18, Z19, Z21, Z22, Z26, Z27, or Z28 is expressed as having a loss of imprinting and/or a copy number variation in the cell nuclei in the lymphatic tissue and/or cell sample, the sample will be determined as a lymphoma tissue and/or cell sample.

In one embodiment of this application, a colorectal tissue and/or cell sample is subjected to an analysis of the expression of any one, or a combination of at least two, of the imprinted genes Z19, Z20, Z21, Z22, Z26, and Z27.

If the imprinted gene Z19, Z20, Z21, Z22, Z26, or Z27 is not expressed or is expressed as normal in the cell nuclei in the colorectal tissue and/or cell sample, the sample will be determined as a healthy colorectal tissue and/or cell sample.

If, however, the imprinted gene Z19, Z20, Z21, Z22, Z26, or Z27 is expressed as having a loss of imprinting and/or a copy number variation in the cell nuclei in the colorectal tissue and/or cell sample, the sample will be determined as a colorectal cancer tissue and/or cell sample.

In one embodiment of this application, a skin tissue and/or cell sample is subjected to an analysis of the expression of any one, or a combination of at least two, of the imprinted genes Z18, Z19, Z21, Z22, Z25, and Z28.

If the imprinted gene Z18, Z19, Z21, Z22, Z25, or Z28 is not expressed or is expressed as normal in the cell nuclei in the skin tissue and/or cell sample, the sample will be determined as a healthy skin tissue and/or cell sample.

If, however, the imprinted gene Z18, Z19, Z21, Z22, Z25, or Z28 is expressed as having a loss of imprinting and/or a copy number variation in the cell nuclei in the skin tissue and/or cell sample, the sample will be determined as a skin cancer tissue and/or cell sample.

In one embodiment of this application, a lung tissue and/or cell sample is subjected to an analysis of the expression of any one, or a combination of at least two, of the imprinted genes Z18, Z19, Z21, Z22, Z25, Z26, Z27, and Z28.

If the imprinted gene Z18, Z19, Z21, Z22, Z25, Z26, Z27, or Z28 is not expressed or is expressed as normal in the cell nuclei in the lung tissue and/or cell sample, the sample will be determined as a healthy lung tissue and/or cell sample.

If, however, the imprinted gene Z18, Z19, Z21, Z22, Z25, Z26, Z27, or Z28 is expressed as having a loss of imprinting and/or a copy number variation in the cell nuclei in the lung tissue and/or cell sample, the sample will be determined as a lung cancer tissue and/or cell sample.

In its fourth aspect, this application provides a method for detecting the degree of benignity/malignancy of a tumor, and the method includes the steps of:

(1) sampling, in which step a sample is obtained;

(2) probe designing, in which step a probe specific to the sequence of an imprinted gene to be detected is designed;

(3) in-situ hybridization, in which step in-situ hybridization is performed between the probe and the sample; and

(4) signal analysis, in which step expression of the imprinted gene is analyzed through microscopic imaging;

wherein an analysis unit carries out tumor diagnosis by analyzing how the imprinted gene(s) of a tumor marker stated in relation to the first aspect is/are expressed in the sample.

Preferably, the sample is sourced from tissue and/or cells.

Preferably, the in-situ hybridization uses an RNAscope in-situ hybridization method.

Preferably, the RNAscope in-situ hybridization method uses a single- or multiple-channel chromogenic reagent kit or a single- or multiple-channel fluorescent reagent kit, preferably a single-channel red/brown chromogenic reagent kit or a multiple-channel fluorescent reagent kit.

In its fifth aspect, this application provides a use of the tumor marker stated in relation to the first aspect, of the tumor diagnosis model stated in relation to the second aspect, or of the tumor diagnosis device stated in relation to the third aspect in preparing a drug and/or a reagent for use in diagnosing, screening, or treating a tumor.

This application has the following advantageous effects over the prior art:

(1) The imprinted genes in this application are not expressed or are expressed as normal in normal tissue or cells, show a loss of imprinting or a copy number variation in a large number of cells in cancer tissue, and therefore have significant diagnostic sensitivity when used as tumor markers.

(2) The imprinted gene-based tumor markers in this application can provide guidance on tumor treatment, facilitate early diagnosis of tumors, help increase patients' survival rates, and thus contribute greatly to saving patients' lives.

(3) The detection method in this application is different from immunohistochemistry, DNA sequencing, DNA methylation analysis, and fluorescent in-situ hybridization (FISH) in that it can reduce false positives and other negative effects while providing important guidance on the treatment and medication of tumors.

### BRIEF DESCRIPTION OF THE SEVERAL VIEWS OF THE DRAWINGS

FIG. 1 shows how 14 imprinted genes are expressed in bile duct tissue samples, with FIG. 1(a) showing how the 14 imprinted genes are expressed in a healthy bile duct tissue sample, and FIG. 1(b) showing how the 14 imprinted genes are expressed in a cholangiocarcinoma tissue sample;

FIG. 2 shows how 19 imprinted genes are expressed in brain tissue samples, with FIG. 2(a) showing how the 19 imprinted genes are expressed in a healthy brain tissue sample, and FIG. 2(b) showing how the 19 imprinted genes are expressed in a brain cancer tissue sample;

FIG. 3 shows how 17 imprinted genes are expressed in kidney tissue samples, with FIG. 3(a) showing how the 17 imprinted genes are expressed in a healthy kidney tissue sample, and FIG. 3(b) showing how the 17 imprinted genes are expressed in a kidney cancer tissue sample;

FIG. 4 shows how 19 imprinted genes are expressed in bone tissue samples, with FIG. 4(a) showing how the 19 imprinted genes are expressed in a healthy bone tissue sample, and FIG. 4(b) showing how the 19 imprinted genes are expressed in a bone cancer tissue sample;

FIG. 5 shows how 17 imprinted genes are expressed in cervical tissue samples, with FIG. 5(a) showing how the 17 imprinted genes are expressed in a healthy cervical tissue sample, and FIG. 5(b) showing how the 17 imprinted genes are expressed in a cervical cancer tissue sample;

FIG. 6 shows how 24 imprinted genes are expressed in nasopharyngeal tissue samples, with FIG. 6(a) showing how the 24 imprinted genes are expressed in a healthy nasopharyngeal tissue sample, and FIG. 6(b) showing how the 24 imprinted genes are expressed in a nasopharyngeal carcinoma tissue sample;

FIG. 7 shows how 16 imprinted genes are expressed in laryngeal tissue samples, with FIG. 7(a) showing how the 16 imprinted genes are expressed in a healthy laryngeal tissue sample, and FIG. 7(b) showing how the 16 imprinted genes are expressed in a laryngeal cancer tissue sample;

FIG. 8 shows how 8 imprinted genes are expressed in prostate tissue samples, with FIG. 8(a) showing how the 8 imprinted genes are expressed in a healthy prostate tissue sample, and FIG. 8(b) showing how the 8 imprinted genes are expressed in a prostate cancer tissue sample;

FIG. 9 shows how 7 imprinted genes are expressed in gastric tissue samples, with FIG. 9(a) showing how the 7 imprinted genes are expressed in a healthy gastric tissue sample, and FIG. 9(b) showing how the 7 imprinted genes are expressed in a gastric carcinoma tissue sample;

FIG. 10 shows how 7 imprinted genes are expressed in esophageal tissue samples, with FIG. 10(a) showing how the 7 imprinted genes are expressed in a healthy esophageal tissue sample, and FIG. 10(b) showing how the 7 imprinted genes are expressed in an esophageal cancer tissue sample;

FIG. 11 shows how 5 imprinted genes are expressed in mammary tissue samples, with FIG. 11(a) showing how the 5 imprinted genes are expressed in a healthy mammary tissue sample, and FIG. 11(b) showing how the 5 imprinted genes are expressed in a mammary cancer tissue sample;

FIG. 12 shows how 5 imprinted genes are expressed in thyroid tissue samples, with FIG. 12(a) showing how the 5 imprinted genes are expressed in a healthy thyroid tissue sample, and FIG. 12(b) showing how the 5 imprinted genes are expressed in a thyroid cancer tissue sample;

FIG. 13 shows how 8 imprinted genes are expressed in bladder tissue samples, with FIG. 13(a) showing how the 8 imprinted genes are expressed in a healthy bladder tissue sample, and FIG. 13(b) showing how the 8 imprinted genes are expressed in a bladder cancer tissue sample;

FIG. 14 shows how 7 imprinted genes are expressed in liver tissue samples, with FIG. 14(a) showing how the 7 imprinted genes are expressed in a healthy liver tissue sample, and FIG. 14(b) showing how the 7 imprinted genes are expressed in a liver cancer tissue sample;

FIG. 15 shows how 9 imprinted genes are expressed in pancreatic tissue samples, with FIG. 15(a) showing how the 9 imprinted genes are expressed in a healthy pancreatic tissue sample, and FIG. 15(b) showing how the 9 imprinted genes are expressed in a pancreatic cancer tissue sample;

FIG. 16 shows how 7 imprinted genes are expressed in lymphatic tissue samples, with FIG. 16(a) showing how the 7 imprinted genes are expressed in a healthy lymphatic tissue sample, and FIG. 16(b) showing how the 7 imprinted genes are expressed in a lymphoma tissue sample;

FIG. 17 shows how 6 imprinted genes are expressed in colorectal tissue samples, with FIG. 17(a) showing how the 6 imprinted genes are expressed in a healthy colorectal tissue sample, and FIG. 17(b) showing how the 6 imprinted genes are expressed in a colorectal cancer tissue sample;

FIG. 18 shows how 6 imprinted genes are expressed in skin tissue samples, with FIG. 18(a) showing how the 6 imprinted genes are expressed in a healthy skin tissue sample, and FIG. 18(b) showing how the 6 imprinted genes are expressed in a skin cancer tissue sample; and

FIG. 19 shows how 8 imprinted genes are expressed in lung tissue samples, with FIG. 19(a) showing how the 8 imprinted genes are expressed in a healthy lung tissue sample, and FIG. 19(b) showing how the 8 imprinted genes are expressed in a lung cancer tissue sample.

### DETAILED DESCRIPTION OF THE INVENTION

To further explain the technical means used in this application and its effects, certain embodiments are detailed below with reference to the accompanying drawings. It should be understood that the embodiments described herein serve only to expound, but not to limit the scope of, the subject matter of this application.

While some of the techniques or conditions used in the embodiments may not be specified herein, those techniques or conditions can be implemented according to the corresponding description in literature in the same technical field as this application or according to the corresponding product manuals. Any reagent or instrument that was used in the embodiments but whose manufacturer is not specified herein is a conventional product commercially available through regular marketing channels.

### Embodiment 1: Imprinted Gene Analysis on Cholangiocarcinoma Samples

(1) Healthy bile duct tissue and cholangiocarcinoma tissue were obtained and placed in 10% neutral buffered formalin in order to be fixed, lest the RNA degrade. After fixing for 24 hours and embedment in paraffin (FFPE), the embedded tissues were cut into 10 μm-thick sections, which were loaded onto positively charged slides. The slides were then baked in a 40° C. oven for at least 3 hours.

(2) The sample processing methods of RNAscope were used to dewax the sections, block the activity of the endogenous peroxidase in the samples, permeabilize the tissues, and expose the RNA molecules.

(3) Probe design: Specific probes were designed according to the sequences of the imprinted genes Z1, Z2, Z8, Z10, N11, Z14, N16, Z19, Z20, Z21, Z22, Z25, Z26, and Z27.

(4) RNAscope reagent kits were used to perform in-situ hybridization between the probes in step (3) and the samples.

(5) After staining with hematoxylin for signal amplification, expression of the imprinted genes was analyzed via microscopic imaging.

In this embodiment, expression of the aforesaid imprinted genes included an imprinted gene not being expressed, the imprinted gene being expressed as normal, the imprinted gene being expressed as having a loss of imprinting, and the imprinted gene being expressed as having a copy number variation, wherein: an imprinted gene was determined as not being expressed when no imprinted gene mark was present in the nucleus of a sample cell, an imprinted gene was determined as being expressed as normal when only one imprinted gene mark was present in the nucleus of a sample cell, an imprinted gene was determined as having a loss of imprinting when two imprinted gene marks were present in the nucleus of a sample cell, and an imprinted gene was determined as having a copy number variation when more than two imprinted gene marks were present in the nucleus of a sample cell.

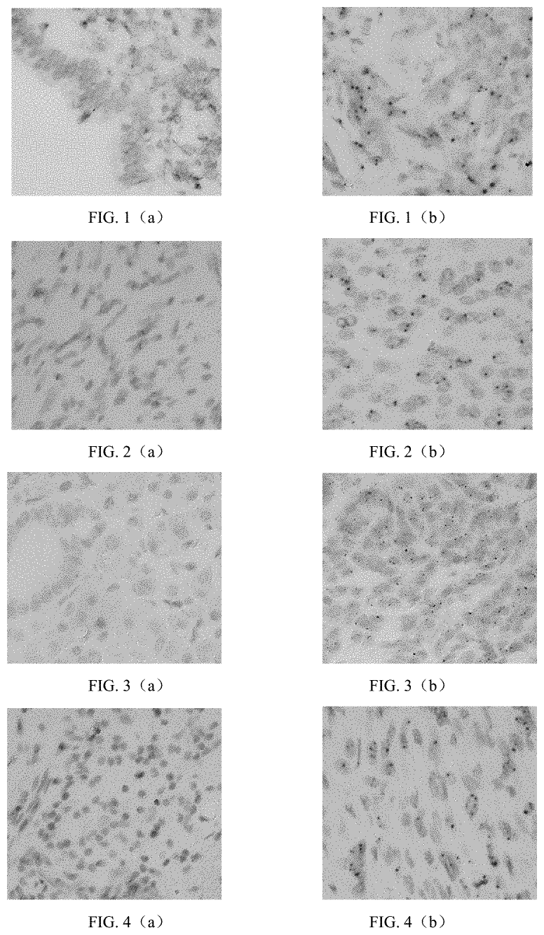

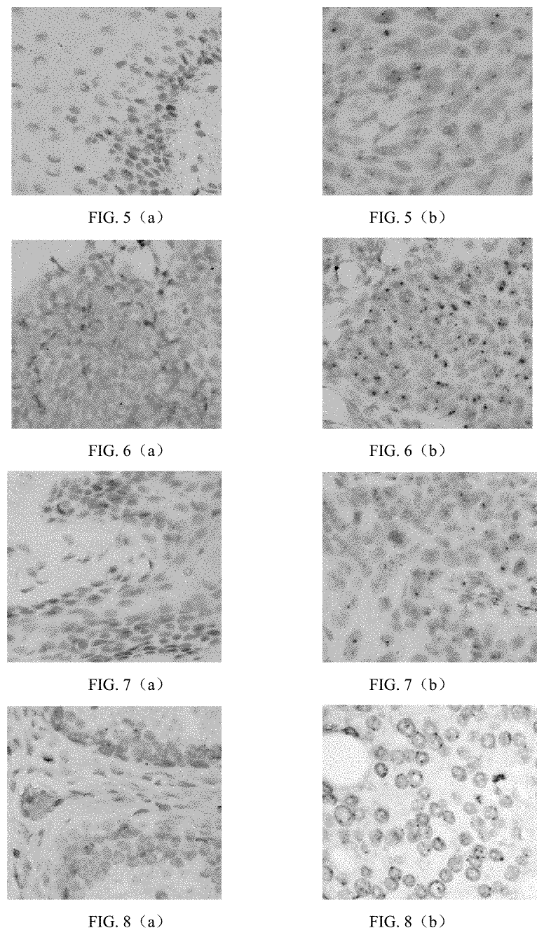

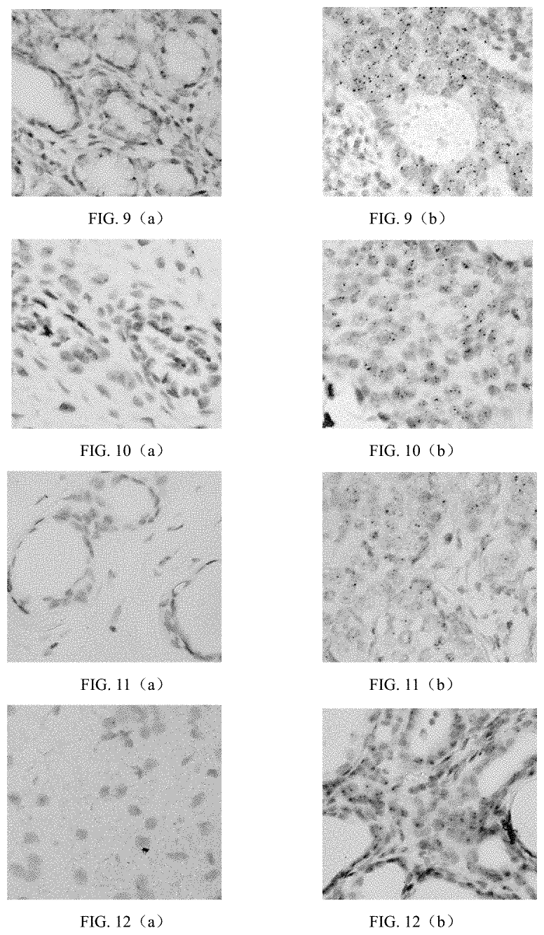

As shown in FIG. 1(a), the 14 imprinted genes were not expressed or were expressed as normal in a healthy bile duct tissue sample, with only a small number of individual cells showing a loss of imprinting or a copy number variation. In a cholangiocarcinoma tissue sample, however, loss of imprinting and copy number variation were found in a large number of cells as shown in FIG. 1(b).

### Embodiment 2: Imprinted Gene Analysis on Brain Cancer Samples

Healthy brain tissue samples and brain cancer tissue samples were obtained, and FFPE sections of the samples were prepared. Specific probes were designed according to the sequences of the imprinted genes Z1, Z2, Z4, Z5, Z8, Z9, Z10, N11, Z13, Z14, N16, Z17, Z19, Z21, Z22, Z23, Z26, Z27, and Z28. The remaining steps of the analysis were the same as those in embodiment 1.

As shown in FIG. 2(a), the 19 imprinted genes were not expressed or were expressed as normal in a healthy brain tissue sample, with only a small number of individual cells showing a loss of imprinting or a copy number variation. In a brain cancer tissue sample, however, loss of imprinting and copy number variation were found in a large number of cells as shown in FIG. 2(b).

### Embodiment 3: Imprinted Gene Analysis on Kidney Cancer Samples

Healthy kidney tissue samples and kidney cancer tissue samples were obtained, and FFPE sections of the samples were prepared. Specific probes were designed according to the sequences of the imprinted genes Z1, Z4, Z6, Z8, Z10, N11, Z13, N16, Z17, Z18, Z19, Z20, Z21, Z25, Z26, Z27, and Z28. The remaining steps of the analysis were the same as those in embodiment 1.

As shown in FIG. 3(a), the 17 imprinted genes were not expressed or were expressed as normal in a healthy kidney tissue sample, with only a small number of individual cells showing a loss of imprinting or a copy number variation. In a kidney cancer tissue sample, however, loss of imprinting and copy number variation were found in a large number of cells as shown in FIG. 3(b).

### Embodiment 4: Imprinted Gene Analysis on Bone Cancer Samples

Healthy bone tissue samples and bone cancer tissue samples were obtained, and FFPE sections of the samples were prepared. Specific probes were designed according to the sequences of the imprinted genes Z1, Z2, Z3, Z6, Z8, Z9, Z10, N11, Z13, N16, Z18, Z19, Z20, Z21, Z22, Z25, Z26, Z27, and Z28. The remaining steps of the analysis were the same as those in embodiment 1.

As shown in FIG. 4(a), the 19 imprinted genes were not expressed or were expressed as normal in a healthy bone tissue sample, with only a small number of individual cells showing a loss of imprinting or a copy number variation. In a bone cancer tissue sample, however, loss of imprinting and copy number variation were found in a large number of cells as shown in FIG. 4(b).

### Embodiment 5: Imprinted Gene Analysis on Cervical Cancer Samples

Healthy cervical tissue samples and cervical cancer tissue samples were obtained, and FFPE sections of the samples were prepared. Specific probes were designed according to the sequences of the imprinted genes Z1, Z3, Z5, Z8, Z9, Z10, N11, N16, Z17, Z19, Z20, Z21, Z22, Z23, Z26, Z27, and Z28. The remaining steps of the analysis were the same as those in embodiment 1.

As shown in FIG. 5(a), the 17 imprinted genes were not expressed or were expressed as normal in a healthy cervical tissue sample, with only a small number of individual cells showing a loss of imprinting or a copy number variation. In a cervical cancer tissue sample, however, loss of imprinting and copy number variation were found in a large number of cells as shown in FIG. 5(b).

### Embodiment 6: Imprinted Gene Analysis on Nasopharyngeal Carcinoma Samples

Healthy nasopharyngeal tissue samples and nasopharyngeal carcinoma tissue samples were obtained, and FFPE sections of the samples were prepared. Specific probes were designed according to the sequences of the imprinted genes Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, and Z28. The remaining steps of the analysis were the same as those in embodiment 1.

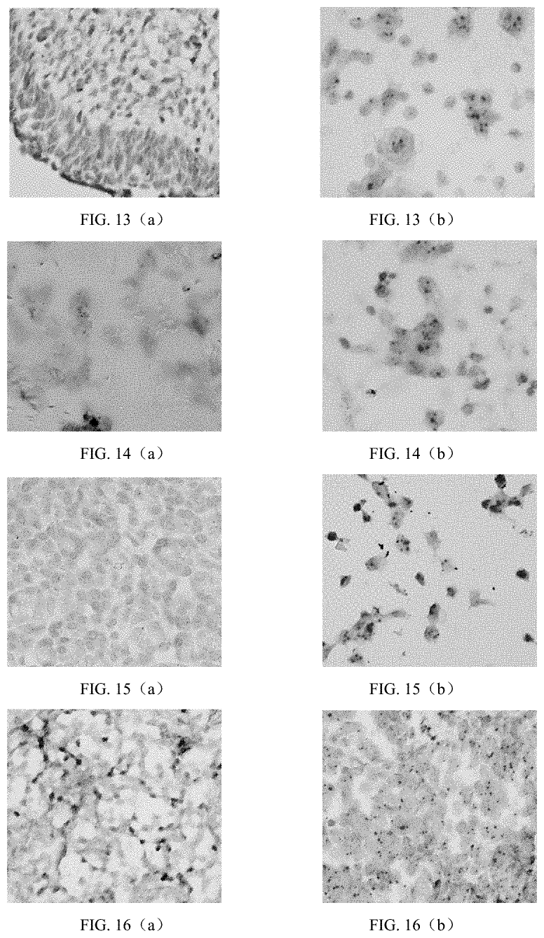

As shown in FIG. 6(a), the 24 imprinted genes were not expressed or were expressed as normal in a healthy nasopharyngeal tissue sample, with only a small number of individual cells showing a loss of imprinting or a copy number variation. In a nasopharyngeal carcinoma tissue sample, however, loss of imprinting and copy number variation were found in a large number of cells as shown in FIG. 6(b).

### Embodiment 7: Imprinted Gene Analysis on Laryngeal Cancer Samples

Healthy laryngeal tissue samples and laryngeal cancer tissue samples were obtained, and FFPE sections of the samples were prepared. Specific probes were designed according to the sequences of the imprinted genes Z1, Z3, Z6, Z8, N11, Z15, N16, Z17, Z18, Z19, Z21, Z22, Z25, Z26, Z27, and Z28. The remaining steps of the analysis were the same as those in embodiment 1.

As shown in FIG. 7(a), the 16 imprinted genes were not expressed or were expressed as normal in a healthy laryngeal tissue sample, with only a small number of individual cells showing a loss of imprinting or a copy number variation. In a laryngeal cancer tissue sample, however, loss of imprinting and copy number variation were found in a large number of cells as shown in FIG. 7(b).

### Embodiment 8: Imprinted Gene Analysis on Prostate Cancer Samples

Healthy prostate tissue samples and prostate cancer tissue samples were obtained, and FFPE sections of the samples were prepared. Specific probes were designed according to the sequences of the imprinted genes Z18, Z19, Z20, Z21, Z25, Z26, Z27, and Z28. The remaining steps of the analysis were the same as those in embodiment 1.

As shown in FIG. 8(a), the 8 imprinted genes were not expressed or were expressed as normal in a healthy prostate tissue sample, with only a small number of individual cells showing a loss of imprinting or a copy number variation. In a prostate cancer tissue sample, however, loss of imprinting and copy number variation were found in a large number of cells as shown in FIG. 8(b).

### Embodiment 9: Imprinted Gene Analysis on Gastric Carcinoma Samples

Healthy gastric tissue samples and gastric carcinoma tissue samples were obtained, and FFPE sections of the samples were prepared. Specific probes were designed according to the sequences of the imprinted genes Z18, Z19, Z20, Z21, Z22, Z26, and Z27. The remaining steps of the analysis were the same as those in embodiment 1.

As shown in FIG. 9(a), the 7 imprinted genes were not expressed or were expressed as normal in a healthy gastric tissue sample, with only a small number of individual cells showing a loss of imprinting or a copy number variation. In a gastric carcinoma tissue sample, however, loss of imprinting and copy number variation were found in a large number of cells as shown in FIG. 9(b).

### Embodiment 10: Imprinted Gene Analysis on Esophageal Cancer Samples

Healthy esophageal tissue samples and esophageal cancer tissue samples were obtained, and FFPE sections of the samples were prepared. Specific probes were designed according to the sequences of the imprinted genes Z18, Z19, Z21, Z22, Z26, Z27, and Z28. The remaining steps of the analysis were the same as those in embodiment 1.

As shown in FIG. 10(a), the 7 imprinted genes were not expressed or were expressed as normal in a healthy esophageal tissue sample, with only a small number of individual cells showing a loss of imprinting or a copy number variation. In an esophageal cancer tissue sample, however, loss of imprinting and copy number variation were found in a large number of cells as shown in FIG. 10(b).

### Embodiment 11: Imprinted Gene Analysis on Mammary Cancer Samples

Healthy mammary tissue samples and mammary cancer tissue samples were obtained, and FFPE sections of the samples were prepared. Specific probes were designed according to the sequences of the imprinted genes Z18, Z19, Z21, Z22, and Z27. The remaining steps of the analysis were the same as those in embodiment 1.

As shown in FIG. 11(a), the 5 imprinted genes were not expressed or were expressed as normal in a healthy mammary tissue sample, with only a small number of individual cells showing a loss of imprinting or a copy number variation. In a mammary cancer tissue sample, however, loss of imprinting and copy number variation were found in a large number of cells as shown in FIG. 11(b).

### Embodiment 12: Imprinted Gene Analysis on Thyroid Cancer Samples

Healthy thyroid tissue samples and thyroid cancer tissue samples were obtained, and FFPE sections of the samples were prepared. Specific probes were designed according to the sequences of the imprinted genes Z18, Z19, Z20, Z21, and Z23. The remaining steps of the analysis were the same as those in embodiment 1.

As shown in FIG. 12(a), the 5 imprinted genes were not expressed or were expressed as normal in a healthy thyroid tissue sample, with only a small number of individual cells showing a loss of imprinting or a copy number variation. In a thyroid cancer tissue sample, however, loss of imprinting and copy number variation were found in a large number of cells as shown in FIG. 12(b).

### Embodiment 13: Imprinted Gene Analysis on Bladder Cancer Samples

Healthy bladder tissue samples and bladder cancer tissue samples were obtained, and FFPE sections of the samples were prepared. Specific probes were designed according to the sequences of the imprinted genes Z18, Z19, Z20, Z21, Z22, Z26, Z27, and Z28. The remaining steps of the analysis were the same as those in embodiment 1.

As shown in FIG. 13(a), the 8 imprinted genes were not expressed or were expressed as normal in a healthy bladder tissue sample, with only a small number of individual cells showing a loss of imprinting or a copy number variation. In a bladder cancer tissue sample, however, loss of imprinting and copy number variation were found in a large number of cells as shown in FIG. 13(b).

### Embodiment 14: Imprinted Gene Analysis on Liver Cancer Samples

Healthy liver tissue samples and liver cancer tissue samples were obtained, and FFPE sections of the samples were prepared. Specific probes were designed according to the sequences of the imprinted genes Z17, Z19, Z21, Z22, Z26, Z27, and Z28. The remaining steps of the analysis were the same as those in embodiment 1.

As shown in FIG. 14(a), the 7 imprinted genes were not expressed or were expressed as normal in a healthy liver tissue sample, with only a small number of individual cells showing a loss of imprinting or a copy number variation. In a liver cancer tissue sample, however, loss of imprinting and copy number variation were found in a large number of cells as shown in FIG. 14(b).

### Embodiment 15: Imprinted Gene Analysis on Pancreatic Cancer Samples

Healthy pancreatic tissue samples and pancreatic cancer tissue samples were obtained, and FFPE sections of the samples were prepared. Specific probes were designed according to the sequences of the imprinted genes Z17, Z18, Z19, Z20, Z21, Z22, Z26, Z27, and Z28. The remaining steps of the analysis were the same as those in embodiment 1.

As shown in FIG. 15(a), the 9 imprinted genes were not expressed or were expressed as normal in a healthy pancreatic tissue sample, with only a small number of individual cells showing a loss of imprinting or a copy number variation. In a pancreatic cancer tissue sample, however, loss of imprinting and copy number variation were found in a large number of cells as shown in FIG. 15(b).

### Embodiment 16: Imprinted Gene Analysis on Lymphoma Samples

Healthy lymphatic tissue samples and lymphoma tissue samples were obtained, and FFPE sections of the samples were prepared. Specific probes were designed according to the sequences of the imprinted genes Z18, Z19, Z21, Z22, Z26, Z27, and Z28. The remaining steps of the analysis were the same as those in embodiment 1.

As shown in FIG. 16(a), the 7 imprinted genes were not expressed or were expressed as normal in a healthy lymphatic tissue sample, with only a small number of individual cells showing a loss of imprinting or a copy number variation. In a lymphoma tissue sample, however, loss of imprinting and copy number variation were found in a large number of cells as shown in FIG. 16(b).

### Embodiment 17: Imprinted Gene Analysis on Colorectal Cancer Samples

Healthy colorectal tissue samples and colorectal cancer tissue samples were obtained, and FFPE sections of the samples were prepared. Specific probes were designed according to the sequences of the imprinted genes Z19, Z20, Z21, Z22, Z26, and Z27. The remaining steps of the analysis were the same as those in embodiment 1.

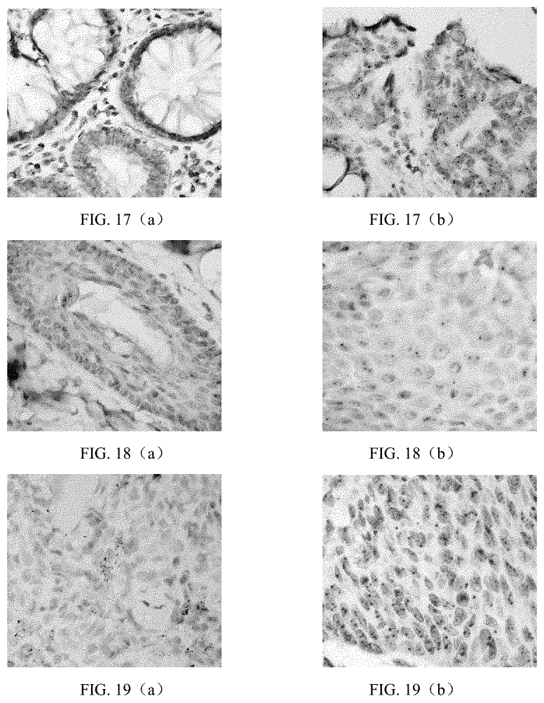

As shown in FIG. 17(a), the 6 imprinted genes were not expressed or were expressed as normal in a healthy colorectal tissue sample, with only a small number of individual cells showing a loss of imprinting or a copy number variation. In a colorectal cancer tissue sample, however, loss of imprinting and copy number variation were found in a large number of cells as shown in FIG. 17(b).

### Embodiment 18: Imprinted Gene Analysis on Skin Cancer Samples

Healthy skin tissue samples and skin cancer tissue samples were obtained, and FFPE sections of the samples were prepared. Specific probes were designed according to the sequences of the imprinted genes Z18, Z19, Z21, Z22, Z25, and Z28. The remaining steps of the analysis were the same as those in embodiment 1.

As shown in FIG. 18(a), the 6 imprinted genes were not expressed or were expressed as normal in a healthy skin tissue sample, with only a small number of individual cells showing a loss of imprinting or a copy number variation. In a skin cancer tissue sample, however, loss of imprinting and copy number variation were found in a large number of cells as shown in FIG. 18(b).

### Embodiment 19: Imprinted Gene Analysis on Lung Cancer Samples

Healthy lung tissue samples and lung cancer tissue samples were obtained, and FFPE sections of the samples were prepared. Specific probes were designed according to the sequences of the imprinted genes Z18, Z19, Z21, Z22, Z25, Z26, Z27, and Z28. The remaining steps of the analysis were the same as those in embodiment 1.

As shown in FIG. 19(a), the 8 imprinted genes were not expressed or were expressed as normal in a healthy lung tissue sample, with only a small number of individual cells showing a loss of imprinting or a copy number variation. In a lung cancer tissue sample, however, loss of imprinting and copy number variation were found in a large number of cells as shown in FIG. 19(b).

According to the above, the imprinted genes in this application are not expressed or are expressed as normal in normal tissue or cells, show a loss of imprinting or a copy number variation in a large number of cells in cancer tissue, and therefore have significant diagnostic sensitivity when used as tumor markers. Those imprinted gene-based tumor markers can provide guidance on tumor treatment, facilitate early diagnosis of tumors, help increase patients' survival rates, and thus contribute greatly to saving patients' lives.

The applicant would like to point out that, while the method in this application has been described in detail by way of the foregoing embodiments, this application is not limited to the method detailed above; in other words, implementation of the subject matter of this application does not necessarily depend on the method detailed above. As would be understood by a person skilled in the art, any improvement made to this application, any equivalent substitution of, or the addition of any auxiliary ingredient to, the raw materials used in the product of this application, and any specific method chosen to implement the subject matter of this application shall fall within the scope of this application and of the patent protection sought by the applicant.

1. A tumor marker, comprising any one, or a combination of at least two, of imprinted genes Z17, Z18, Z19, Z20, Z21, Z22, Z23, Z25, Z26, Z27, and Z28;
wherein the imprinted gene Z17 is Aldh111, the imprinted gene Z18 is Gata3, the imprinted gene Z19 is Hm13, the imprinted gene Z20 is Kcnq1, the imprinted gene Z21 is Nhp211, the imprinted gene Z22 is Pon2, the imprinted gene Z23 is Rasgrf1, the imprinted gene Z25 is Tfpi2, the imprinted gene Z26 is Trappc9, the imprinted gene Z27 is Ube3a, and the imprinted gene Z28 is Zic1.

2. The tumor marker of claim 1, wherein the tumor marker further comprises any one, or a combination of at least two, of imprinted genes Z1, Z2, Z3, Z4, Z5, Z6, Z8, Z9, Z10, N11, Z12, Z13, Z14, Z15, and N16;
wherein the imprinted gene Z1 is Gnas, the imprinted gene Z2 is Igf2, the imprinted gene Z3 is Peg10, the imprinted gene Z4 is Igf2r, the imprinted gene Z5 is Mest, the imprinted gene Z6 is Plagl1, the imprinted gene Z8 is Dcn, the imprinted gene Z9 is Dlk1, the imprinted gene Z10 is Gatm, the imprinted gene N11 is Grb10, the imprinted gene Z12 is Peg3, the imprinted gene Z13 is Sgce, the imprinted gene Z14 is Slc38a4, the imprinted gene Z15 is Diras3, and the imprinted gene N16 is Snrpn/Snurf.

3. The tumor marker of claim 1 or 2, wherein said tumor comprises any one, or a combination of at least two, of cholangiocarcinoma, brain cancer, kidney cancer, bone cancer, cervical cancer, nasopharyngeal carcinoma, and laryngeal cancer.

4. The tumor marker of claim 3, wherein said tumor further comprises any one, or a combination of at least two, of prostate cancer, gastric carcinoma, esophageal cancer, mammary cancer, thyroid cancer, bladder cancer, liver cancer, pancreatic cancer, lymphoma, colorectal cancer, skin cancer, and lung cancer.

5. The tumor marker of any of claims 2 to 4, wherein:
the tumor marker is a marker for cholangiocarcinoma and comprises any one, or a combination of at least two, of the imprinted genes Z1, Z2, Z8, Z10, N11, Z14, N16, Z19, Z20, Z21, Z22, Z25, Z26, and Z27; or
the tumor marker is a marker for brain cancer and comprises any one, or a combination of at least two, of the imprinted genes Z1, Z2, Z4, Z5, Z8, Z9, Z10, N11, Z13, Z14, N16, Z17, Z19, Z21, Z22, Z23, Z26, Z27, and Z28; or
the tumor marker is a marker for kidney cancer and comprises any one, or a combination of at least two, of the imprinted genes Z1, Z4, Z6, Z8, Z10, N11, Z13, N16, Z17, Z18, Z19, Z20, Z21, Z25, Z26, Z27, and Z28; or
the tumor marker is a marker for bone cancer and comprises any one, or a combination of at least two, of the imprinted genes Z1, Z2, Z3, Z6, Z8, Z9, Z10, N11, Z13, N16, Z18, Z19, Z20, Z21, Z22, Z25, Z26, Z27, and Z28; or
the tumor marker is a marker for cervical cancer and comprises any one, or a combination of at least two, of the imprinted genes Z1, Z3, Z5, Z8, Z9, Z10, N11, N16, Z17, Z19, Z20, Z21, Z22, Z23, Z26, Z27, and Z28; or
the tumor marker is a marker for nasopharyngeal carcinoma and comprises any one, or a combination of at least two, of the imprinted genes Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, and Z28; or
the tumor marker is a marker for laryngeal cancer and comprises any one, or a combination of at least two, of the imprinted genes Z1, Z3, Z6, Z8, N11, Z15, N16, Z17, Z18, Z19, Z21, Z22, Z25, Z26, Z27, and Z28; or
the tumor marker is a marker for prostate cancer and comprises any one, or a combination of at least two, of the imprinted genes Z18, Z19, Z20, Z21, Z25, Z26, Z27, and Z28; or
the tumor marker is a marker for gastric carcinoma and comprises any one, or a combination of at least two, of the imprinted genes Z18, Z19, Z20, Z21, Z22, Z26, and Z27; or
the tumor marker is a marker for esophageal cancer and comprises any one, or a combination of at least two, of the imprinted genes Z18, Z19, Z21, Z22, Z26, Z27, and Z28; or
the tumor marker is a marker for mammary cancer and comprises any one, or a combination of at least two, of the imprinted genes Z18, Z19, Z21, Z22, and Z27; or
the tumor marker is a marker for thyroid cancer and comprises any one, or a combination of at least two, of the imprinted genes Z18, Z19, Z20, Z21, and Z23; or
the tumor marker is a marker for bladder cancer and comprises any one, or a combination of at least two, of the imprinted genes Z18, Z19, Z20, Z21, Z22, Z26, Z27, and Z28; or
the tumor marker is a marker for liver cancer and comprises any one, or a combination of at least two, of the imprinted genes Z17, Z19, Z21, Z22, Z26, Z27, and Z28; or
the tumor marker is a marker for pancreatic cancer and comprises any one, or a combination of at least two, of the imprinted genes Z17, Z18, Z19, Z20, Z21, Z22, Z26, Z27, and Z28; or
the tumor marker is a marker for lymphoma and comprises any one, or a combination of at least two, of the imprinted genes Z18, Z19, Z21, Z22, Z26, Z27, and Z28; or
the tumor marker is a marker for colorectal cancer and comprises any one, or a combination of at least two, of the imprinted genes Z19, Z20, Z21, Z22, Z26, and Z27; or
the tumor marker is a marker for skin cancer and comprises any one, or a combination of at least two, of the imprinted genes Z18, Z19, Z21, Z22, Z25, and Z28; or
the tumor marker is a marker for lung cancer and comprises any one, or a combination of at least two, of the imprinted genes Z18, Z19, Z21, Z22, Z25, Z26, Z27, and Z28.

6. A tumor diagnosis model for carrying out tumor diagnosis by analyzing expression of the imprinted gene(s) of the tumor marker of any of claims 1 to 5 in a sample;
wherein the expression of the imprinted gene(s) comprises any one, or a combination of at least two, of the imprinted gene(s) not being expressed, the imprinted gene(s) being expressed as normal, the imprinted gene(s) being expressed as having a loss of imprinting, and the imprinted gene(s) being expressed as having a copy number variation.

7. The tumor diagnosis model of claim 6, wherein:
a said imprinted gene is determined as not being expressed when no imprinted gene mark is present in the nucleus of a sample cell;
the imprinted gene is determined as being expressed as normal when only one said imprinted gene mark is present in the nucleus of the sample cell;
the imprinted gene is determined as having a loss of imprinting when two said imprinted gene marks are present in the nucleus of the sample cell; and
the imprinted gene is determined as having a copy number variation when more than two said imprinted gene marks are present in the nucleus of the sample cell.

8. The tumor diagnosis model of claim 6 or 7, wherein the tumor diagnosis model uses a diagnostic method comprising:
(a) subjecting a bile duct tissue and/or cell sample to an analysis of the expression of any one, or a combination of at least two, of the imprinted genes Z1, Z2, Z8, Z10, N11, Z14, N16, Z19, Z20, Z21, Z22, Z25, Z26, and Z27 so that:
if the imprinted gene Z1, Z2, Z8, Z10, N11, Z14, N16, Z19, Z20, Z21, Z22, Z25, Z26, or Z27 is not expressed or is expressed as normal in cell nuclei in the bile duct tissue and/or cell sample, the bile duct tissue and/or cell sample is determined as a healthy bile duct tissue and/or cell sample; and
if the imprinted gene Z1, Z2, Z8, Z10, N11, Z14, N16, Z19, Z20, Z21, Z22, Z25, Z26, or Z27 is expressed as having a loss of imprinting and/or a copy number variation in the cell nuclei in the bile duct tissue and/or cell sample, the bile duct tissue and/or cell sample is determined as a cholangiocarcinoma tissue and/or cell sample;
or
(b) subjecting a brain tissue and/or cell sample to an analysis of the expression of any one, or a combination of at least two, of the imprinted genes Z1, Z2, Z4, Z5, Z8, Z9, Z10, N11, Z13, Z14, N16, Z17, Z19, Z21, Z22, Z23, Z26, Z27, and Z28 so that:
if the imprinted gene Z1, Z2, Z4, Z5, Z8, Z9, Z10, N11, Z13, Z14, N16, Z17, Z19, Z21, Z22, Z23, Z26, Z27, or Z28 is not expressed or is expressed as normal in cell nuclei in the brain tissue and/or cell sample, the brain tissue and/or cell sample is determined as a healthy brain tissue and/or cell sample; and
if the imprinted gene Z1, Z2, Z4, Z5, Z8, Z9, Z10, N11, Z13, Z14, N16, Z17, Z19, Z21, Z22, Z23, Z26, Z27, or Z28 is expressed as having a loss of imprinting and/or a copy number variation in the cell nuclei in the brain tissue and/or cell sample, the brain tissue and/or cell sample is determined as a brain cancer tissue and/or cell sample;
or
(c) subjecting a kidney tissue and/or cell sample to an analysis of the expression of any one, or a combination of at least two, of the imprinted genes Z1, Z4, Z6, Z8, Z10, N11, Z13, N16, Z17, Z18, Z19, Z20, Z21, Z25, Z26, Z27, and Z28 so that:
if the imprinted gene Z1, Z4, Z6, Z8, Z10, N11, Z13, N16, Z17, Z18, Z19, Z20, Z21, Z25, Z26, Z27, or Z28 is not expressed or is expressed as normal in cell nuclei in the kidney tissue and/or cell sample, the kidney tissue and/or cell sample is determined as a healthy kidney tissue and/or cell sample; and
if the imprinted gene Z1, Z4, Z6, Z8, Z10, N11, Z13, N16, Z17, Z18, Z19, Z20, Z21, Z25, Z26, Z27, or Z28 is expressed as having a loss of imprinting and/or a copy number variation in the cell nuclei in the kidney tissue and/or cell sample, the kidney tissue and/or cell sample is determined as a kidney cancer tissue and/or cell sample;
or
(d) subjecting a bone tissue and/or cell sample to an analysis of the expression of any one, or a combination of at least two, of the imprinted genes Z1, Z2, Z3, Z6, Z8, Z9, Z10, N11, Z13, N16, Z18, Z19, Z20, Z21, Z22, Z25, Z26, Z27, and Z28 so that:
if the imprinted gene Z1, Z2, Z3, Z6, Z8, Z9, Z10, N11, Z13, N16, Z18, Z19, Z20, Z21, Z22, Z25, Z26, Z27, or Z28 is not expressed or is expressed as normal in cell nuclei in the bone tissue and/or cell sample, the bone tissue and/or cell sample is determined as a healthy bone tissue and/or cell sample; and
if the imprinted gene Z1, Z2, Z3, Z6, Z8, Z9, Z10, N11, Z13, N16, Z18, Z19, Z20, Z21, Z22, Z25, Z26, Z27, or Z28 is expressed as having a loss of imprinting and/or a copy number variation in the cell nuclei in the bone tissue and/or cell sample, the bone tissue and/or cell sample is determined as a bone cancer tissue and/or cell sample;
or
(e) subjecting a cervical tissue and/or cell sample to an analysis of the expression of any one, or a combination of at least two, of the imprinted genes Z1, Z3, Z5, Z8, Z9, Z10, N11, N16, Z17, Z19, Z20, Z21, Z22, Z23, Z26, Z27, and Z28 so that:
if the imprinted gene Z1, Z3, Z5, Z8, Z9, Z10, N11, N16, Z17, Z19, Z20, Z21, Z22, Z23, Z26, Z27, or Z28 is not expressed or is expressed as normal in cell nuclei in the cervical tissue and/or cell sample, the cervical tissue and/or cell sample is determined as a healthy cervical tissue and/or cell sample; and
if the imprinted gene Z1, Z3, Z5, Z8, Z9, Z10, N11, N16, Z17, Z19, Z20, Z21, Z22, Z23, Z26, Z27, or Z28 is expressed as having a loss of imprinting and/or a copy number variation in the cell nuclei in the cervical tissue and/or cell sample, the cervical tissue and/or cell sample is determined as a cervical cancer tissue and/or cell sample;
or
(f) subjecting a nasopharyngeal tissue and/or cell sample to an analysis of the expression of any one, or a combination of at least two, of the imprinted genes Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, and Z28 so that:
if the imprinted gene Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, or Z28 is not expressed or is expressed as normal in cell nuclei in the nasopharyngeal tissue and/or cell sample, the nasopharyngeal tissue and/or cell sample is determined as a healthy nasopharyngeal tissue and/or cell sample; and
if the imprinted gene Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, or Z28 is expressed as having a loss of imprinting and/or a copy number variation in the cell nuclei in the nasopharyngeal tissue and/or cell sample, the nasopharyngeal tissue and/or cell sample is determined as a nasopharyngeal carcinoma tissue and/or cell sample;
or
(g) subjecting a laryngeal tissue and/or cell sample to an analysis of the expression of any one, or a combination of at least two, of the imprinted genes Z1, Z3, Z6, Z8, N11, Z15, N16, Z17, Z18, Z19, Z21, Z22, Z25, Z26, Z27, and Z28 so that:
if the imprinted gene Z1, Z3, Z6, Z8, N11, Z15, N16, Z17, Z18, Z19, Z21, Z22, Z25, Z26, Z27, or Z28 is not expressed or is expressed as normal in cell nuclei in the laryngeal tissue and/or cell sample, the laryngeal tissue and/or cell sample is determined as a healthy laryngeal tissue and/or cell sample; and
if the imprinted gene Z1, Z3, Z6, Z8, N11, Z15, N16, Z17, Z18, Z19, Z21, Z22, Z25, Z26, Z27, or Z28 is expressed as having a loss of imprinting and/or a copy number variation in the cell nuclei in the laryngeal tissue and/or cell sample, the laryngeal tissue and/or cell sample is determined as a laryngeal cancer tissue and/or cell sample;
or
(h) subjecting a prostate tissue and/or cell sample to an analysis of the expression of any one, or a combination of at least two, of the imprinted genes Z18, Z19, Z20, Z21, Z25, Z26, Z27, and Z28 so that:
if the imprinted gene Z18, Z19, Z20, Z21, Z25, Z26, Z27, or Z28 is not expressed or is expressed as normal in cell nuclei in the prostate tissue and/or cell sample, the prostate tissue and/or cell sample is determined as a healthy prostate tissue and/or cell sample; and
if the imprinted gene Z18, Z19, Z20, Z21, Z25, Z26, Z27, or Z28 is expressed as having a loss of imprinting and/or a copy number variation in the cell nuclei in the prostate tissue and/or cell sample, the prostate tissue and/or cell sample is determined as a prostate cancer tissue and/or cell sample;
or
(i) subjecting a gastric tissue and/or cell sample to an analysis of the expression of any one, or a combination of at least two, of the imprinted genes Z18, Z19, Z20, Z21, Z22, Z26, and Z27 so that:
if the imprinted gene Z18, Z19, Z20, Z21, Z22, Z26, or Z27 is not expressed or is expressed as normal in cell nuclei in the gastric tissue and/or cell sample, the gastric tissue and/or cell sample is determined as a healthy gastric tissue and/or cell sample; and
if the imprinted gene Z18, Z19, Z20, Z21, Z22, Z26, or Z27 is expressed as having a loss of imprinting and/or a copy number variation in the cell nuclei in the gastric tissue and/or cell sample, the gastric tissue and/or cell sample is determined as a gastric carcinoma tissue and/or cell sample;
or
(j) subjecting an esophageal tissue and/or cell sample to an analysis of the expression of any one, or a combination of at least two, of the imprinted genes Z18, Z19, Z21, Z22, Z26, Z27, and Z28 so that:
if the imprinted gene Z18, Z19, Z21, Z22, Z26, Z27, or Z28 is not expressed or is expressed as normal in cell nuclei in the esophageal tissue and/or cell sample, the esophageal tissue and/or cell sample is determined as a healthy esophageal tissue and/or cell sample; and
if the imprinted gene Z18, Z19, Z21, Z22, Z26, Z27, or Z28 is expressed as having a loss of imprinting and/or a copy number variation in the cell nuclei in the esophageal tissue and/or cell sample, the esophageal tissue and/or cell sample is determined as an esophageal cancer tissue and/or cell sample;
or
(k) subjecting a mammary tissue and/or cell sample to an analysis of the expression of any one, or a combination of at least two, of the imprinted genes Z18, Z19, Z21, Z22, and Z27 so that:
if the imprinted gene Z18, Z19, Z21, Z22, or Z27 is not expressed or is expressed as normal in cell nuclei in the mammary tissue and/or cell sample, the mammary tissue and/or cell sample is determined as a healthy mammary tissue and/or cell sample; and
if the imprinted gene Z18, Z19, Z21, Z22, or Z27 is expressed as having a loss of imprinting and/or a copy number variation in the cell nuclei in the mammary tissue and/or cell sample, the mammary tissue and/or cell sample is determined as a mammary cancer tissue and/or cell sample;
or
(l) subjecting a thyroid tissue and/or cell sample to an analysis of the expression of any one, or a combination of at least two, of the imprinted genes Z18, Z19, Z20, Z21, and Z23 so that:
if the imprinted gene Z18, Z19, Z20, Z21, or Z23 is not expressed or is expressed as normal in cell nuclei in the thyroid tissue and/or cell sample, the thyroid tissue and/or cell sample is determined as a healthy thyroid tissue and/or cell sample; and
if the imprinted gene Z18, Z19, Z20, Z21, or Z23 is expressed as having a loss of imprinting and/or a copy number variation in the cell nuclei in the thyroid tissue and/or cell sample, the thyroid tissue and/or cell sample is determined as a thyroid cancer tissue and/or cell sample;
or
(m) subjecting a bladder tissue and/or cell sample to an analysis of the expression of any one, or a combination of at least two, of the imprinted genes Z18, Z19, Z20, Z21, Z22, Z26, Z27, and Z28 so that:
if the imprinted gene Z18, Z19, Z20, Z21, Z22, Z26, Z27, or Z28 is not expressed or is expressed as normal in cell nuclei in the bladder tissue and/or cell sample, the bladder tissue and/or cell sample is determined as a healthy bladder tissue and/or cell sample; and
if the imprinted gene Z18, Z19, Z20, Z21, Z22, Z26, Z27, or Z28 is expressed as having a loss of imprinting and/or a copy number variation in the cell nuclei in the bladder tissue and/or cell sample, the bladder tissue and/or cell sample is determined as a bladder cancer tissue and/or cell sample;
or
(n) subjecting a liver tissue and/or cell sample to an analysis of the expression of any one, or a combination of at least two, of the imprinted genes Z17, Z19, Z21, Z22, Z26, Z27, and Z28 so that:
if the imprinted gene Z17, Z19, Z21, Z22, Z26, Z27, or Z28 is not expressed or is expressed as normal in cell nuclei in the liver tissue and/or cell sample, the liver tissue and/or cell sample is determined as a healthy liver tissue and/or cell sample; and
if the imprinted gene Z17, Z19, Z21, Z22, Z26, Z27, or Z28 is expressed as having a loss of imprinting and/or a copy number variation in the cell nuclei in the liver tissue and/or cell sample, the liver tissue and/or cell sample is determined as a liver cancer tissue and/or cell sample;
or
(o) subjecting a pancreatic tissue and/or cell sample to an analysis of the expression of any one, or a combination of at least two, of the imprinted genes Z17, Z18, Z19, Z20, Z21, Z22, Z26, Z27, and Z28 so that:
if the imprinted gene Z17, Z18, Z19, Z20, Z21, Z22, Z26, Z27, or Z28 is not expressed or is expressed as normal in cell nuclei in the pancreatic tissue and/or cell sample, the pancreatic tissue and/or cell sample is determined as a healthy pancreatic tissue and/or cell sample; and
if the imprinted gene Z17, Z18, Z19, Z20, Z21, Z22, Z26, Z27, or Z28 is expressed as having a loss of imprinting and/or a copy number variation in the cell nuclei in the pancreatic tissue and/or cell sample, the pancreatic tissue and/or cell sample is determined as a pancreatic cancer tissue and/or cell sample;
or
(p) subjecting a lymphatic tissue and/or cell sample to an analysis of the expression of any one, or a combination of at least two, of the imprinted genes Z18, Z19, Z21, Z22, Z26, Z27, and Z28 so that:
if the imprinted gene Z18, Z19, Z21, Z22, Z26, Z27, or Z28 is not expressed or is expressed as normal in cell nuclei in the lymphatic tissue and/or cell sample, the lymphatic tissue and/or cell sample is determined as a healthy lymphatic tissue and/or cell sample; and
if the imprinted gene Z18, Z19, Z21, Z22, Z26, Z27, or Z28 is expressed as having a loss of imprinting and/or a copy number variation in the cell nuclei in the lymphatic tissue and/or cell sample, the lymphatic tissue and/or cell sample is determined as a lymphoma tissue and/or cell sample;
or
(q) subjecting a colorectal tissue and/or cell sample to an analysis of the expression of any one, or a combination of at least two, of the imprinted genes Z19, Z20, Z21, Z22, Z26, and Z27 so that:
if the imprinted gene Z19, Z20, Z21, Z22, Z26, or Z27 is not expressed or is expressed as normal in cell nuclei in the colorectal tissue and/or cell sample, the colorectal tissue and/or cell sample is determined as a healthy colorectal tissue and/or cell sample; and
if the imprinted gene Z19, Z20, Z21, Z22, Z26, or Z27 is expressed as having a loss of imprinting and/or a copy number variation in the cell nuclei in the colorectal tissue and/or cell sample, the colorectal tissue and/or cell sample is determined as a colorectal cancer tissue and/or cell sample;
or
(r) subjecting a skin tissue and/or cell sample to an analysis of the expression of any one, or a combination of at least two, of the imprinted genes Z18, Z19, Z21, Z22, Z25, and Z28 so that:
if the imprinted gene Z18, Z19, Z21, Z22, Z25, or Z28 is not expressed or is expressed as normal in cell nuclei in the skin tissue and/or cell sample, the skin tissue and/or cell sample is determined as a healthy skin tissue and/or cell sample; and
if the imprinted gene Z18, Z19, Z21, Z22, Z25, or Z28 is expressed as having a loss of imprinting and/or a copy number variation in the cell nuclei in the skin tissue and/or cell sample, the skin tissue and/or cell sample is determined as a skin cancer tissue and/or cell sample;
or
(s) subjecting a lung tissue and/or cell sample to an analysis of the expression of any one, or a combination of at least two, of the imprinted genes Z18, Z19, Z21, Z22, Z25, Z26, Z27, and Z28 so that:
if the imprinted gene Z18, Z19, Z21, Z22, Z25, Z26, Z27, or Z28 is not expressed or is expressed as normal in cell nuclei in the lung tissue and/or cell sample, the lung tissue and/or cell sample is determined as a healthy lung tissue and/or cell sample; and
if the imprinted gene Z18, Z19, Z21, Z22, Z25, Z26, Z27, or Z28 is expressed as having a loss of imprinting and/or a copy number variation in the cell nuclei in the lung tissue and/or cell sample, the lung tissue and/or cell sample is determined as a lung cancer tissue and/or cell sample.

9. A tumor diagnosis device, comprising:
a sampling unit for obtaining a sample;
a probe designing unit for designing a probe specific to the sequence of an imprinted gene to be detected;
a detection unit for performing in-situ hybridization between the probe and the sample; and
an analysis unit for analyzing expression of the imprinted gene through microscopic imaging;
wherein the analysis unit carries out tumor diagnosis by analyzing expression of the imprinted gene(s) of the tumor marker of any of claims 1 to 5 in the sample.

10. The tumor diagnosis device of claim 9, wherein the sample is sourced from tissue and/or cells.

11. The tumor diagnosis device of claim 9, wherein the in-situ hybridization uses an RNAscope in-situ hybridization method.

12. The tumor diagnosis device of claim 11, wherein the RNAscope in-situ hybridization method uses a single- or multiple-channel chromogenic reagent kit or a single- or multiple-channel fluorescent reagent kit.

13. The tumor diagnosis device of claim 12, wherein the RNAscope in-situ hybridization method uses a single-channel red/brown chromogenic reagent kit or a multiple-channel fluorescent reagent kit.

14. The tumor diagnosis device of any of claims 9 to 13, wherein the tumor diagnosis device uses an analysis method comprising:
(a) subjecting a bile duct tissue and/or cell sample to an analysis of the expression of any one, or a combination of at least two, of the imprinted genes Z1, Z2, Z8, Z10, N11, Z14, N16, Z19, Z20, Z21, Z22, Z25, Z26, and Z27 so that:
if the imprinted gene Z1, Z2, Z8, Z10, N11, Z14, N16, Z19, Z20, Z21, Z22, Z25, Z26, or Z27 is not expressed or is expressed as normal in cell nuclei in the bile duct tissue and/or cell sample, the bile duct tissue and/or cell sample is determined as a healthy bile duct tissue and/or cell sample; and
if the imprinted gene Z1, Z2, Z8, Z10, N11, Z14, N16, Z19, Z20, Z21, Z22, Z25, Z26, or Z27 is expressed as having a loss of imprinting and/or a copy number variation in the cell nuclei in the bile duct tissue and/or cell sample, the bile duct tissue and/or cell sample is determined as a cholangiocarcinoma tissue and/or cell sample;
or
(b) subjecting a brain tissue and/or cell sample to an analysis of the expression of any one, or a combination of at least two, of the imprinted genes Z1, Z2, Z4, Z5, Z8, Z9, Z10, N11, Z13, Z14, N16, Z17, Z19, Z21, Z22, Z23, Z26, Z27, and Z28 so that:
if the imprinted gene Z1, Z2, Z4, Z5, Z8, Z9, Z10, N11, Z13, Z14, N16, Z17, Z19, Z21, Z22, Z23, Z26, Z27, or Z28 is not expressed or is expressed as normal in cell nuclei in the brain tissue and/or cell sample, the brain tissue and/or cell sample is determined as a healthy brain tissue and/or cell sample; and
if the imprinted gene Z1, Z2, Z4, Z5, Z8, Z9, Z10, N11, Z13, Z14, N16, Z17, Z19, Z21, Z22, Z23, Z26, Z27, or Z28 is expressed as having a loss of imprinting and/or a copy number variation in the cell nuclei in the brain tissue and/or cell sample, the brain tissue and/or cell sample is determined as a brain cancer tissue and/or cell sample;
or
(c) subjecting a kidney tissue and/or cell sample to an analysis of the expression of any one, or a combination of at least two, of the imprinted genes Z1, Z4, Z6, Z8, Z10, N11, Z13, N16, Z17, Z18, Z19, Z20, Z21, Z25, Z26, Z27, and Z28 so that:
if the imprinted gene Z1, Z4, Z6, Z8, Z10, N11, Z13, N16, Z17, Z18, Z19, Z20, Z21, Z25, Z26, Z27, or Z28 is not expressed or is expressed as normal in cell nuclei in the kidney tissue and/or cell sample, the kidney tissue and/or cell sample is determined as a healthy kidney tissue and/or cell sample; and
if the imprinted gene Z1, Z4, Z6, Z8, Z10, N11, Z13, N16, Z17, Z18, Z19, Z20, Z21, Z25, Z26, Z27, or Z28 is expressed as having a loss of imprinting and/or a copy number variation in the cell nuclei in the kidney tissue and/or cell sample, the kidney tissue and/or cell sample is determined as a kidney cancer tissue and/or cell sample;
or
(d) subjecting a bone tissue and/or cell sample to an analysis of the expression of any one, or a combination of at least two, of the imprinted genes Z1, Z2, Z3, Z6, Z8, Z9, Z10, N11, Z13, N16, Z18, Z19, Z20, Z21, Z22, Z25, Z26, Z27, and Z28 so that:
if the imprinted gene Z1, Z2, Z3, Z6, Z8, Z9, Z10, N11, Z13, N16, Z18, Z19, Z20, Z21, Z22, Z25, Z26, Z27, or Z28 is not expressed or is expressed as normal in cell nuclei in the bone tissue and/or cell sample, the bone tissue and/or cell sample is determined as a healthy bone tissue and/or cell sample; and
if the imprinted gene Z1, Z2, Z3, Z6, Z8, Z9, Z10, N11, Z13, N16, Z18, Z19, Z20, Z21, Z22, Z25, Z26, Z27, or Z28 is expressed as having a loss of imprinting and/or a copy number variation in the cell nuclei in the bone tissue and/or cell sample, the bone tissue and/or cell sample is determined as a bone cancer tissue and/or cell sample;
or
(e) subjecting a cervical tissue and/or cell sample to an analysis of the expression of any one, or a combination of at least two, of the imprinted genes Z1, Z3, Z5, Z8, Z9, Z10, N11, N16, Z17, Z19, Z20, Z21, Z22, Z23, Z26, Z27, and Z28 so that:
if the imprinted gene Z1, Z3, Z5, Z8, Z9, Z10, N11, N16, Z17, Z19, Z20, Z21, Z22, Z23, Z26, Z27, or Z28 is not expressed or is expressed as normal in cell nuclei in the cervical tissue and/or cell sample, the cervical tissue and/or cell sample is determined as a healthy cervical tissue and/or cell sample; and
if the imprinted gene Z1, Z3, Z5, Z8, Z9, Z10, N11, N16, Z17, Z19, Z20, Z21, Z22, Z23, Z26, Z27, or Z28 is expressed as having a loss of imprinting and/or a copy number variation in the cell nuclei in the cervical tissue and/or cell sample, the cervical tissue and/or cell sample is determined as a cervical cancer tissue and/or cell sample;
or
(f) subjecting a nasopharyngeal tissue and/or cell sample to an analysis of the expression of any one, or a combination of at least two, of the imprinted genes Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, and Z28 so that:
if the imprinted gene Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, or Z28 is not expressed or is expressed as normal in cell nuclei in the nasopharyngeal tissue and/or cell sample, the nasopharyngeal tissue and/or cell sample is determined as a healthy nasopharyngeal tissue and/or cell sample; and
if the imprinted gene Z1, Z3, Z4, Z5, Z6, Z8, Z9, Z10, N11, Z12, Z13, Z14, N16, Z17, Z18, Z19, Z20, Z21, Z22, Z23, Z25, Z26, Z27, or Z28 is expressed as having a loss of imprinting and/or a copy number variation in the cell nuclei in the nasopharyngeal tissue and/or cell sample, the nasopharyngeal tissue and/or cell sample is determined as a nasopharyngeal carcinoma tissue and/or cell sample;
or
(g) subjecting a laryngeal tissue and/or cell sample to an analysis of the expression of any one, or a combination of at least two, of the imprinted genes Z1, Z3, Z6, Z8, N11, Z15, N16, Z17, Z18, Z19, Z21, Z22, Z25, Z26, Z27, and Z28 so that:
if the imprinted gene Z1, Z3, Z6, Z8, N11, Z15, N16, Z17, Z18, Z19, Z21, Z22, Z25, Z26, Z27, or Z28 is not expressed or is expressed as normal in cell nuclei in the laryngeal tissue and/or cell sample, the laryngeal tissue and/or cell sample is determined as a healthy laryngeal tissue and/or cell sample; and
if the imprinted gene Z1, Z3, Z6, Z8, N11, Z15, N16, Z17, Z18, Z19, Z21, Z22, Z25, Z26, Z27, or Z28 is expressed as having a loss of imprinting and/or a copy number variation in the cell nuclei in the laryngeal tissue and/or cell sample, the laryngeal tissue and/or cell sample is determined as a laryngeal cancer tissue and/or cell sample;
or
(h) subjecting a prostate tissue and/or cell sample to an analysis of the expression of any one, or a combination of at least two, of the imprinted genes Z18, Z19, Z20, Z21, Z25, Z26, Z27, and Z28 so that:
if the imprinted gene Z18, Z19, Z20, Z21, Z25, Z26, Z27, or Z28 is not expressed or is expressed as normal in cell nuclei in the prostate tissue and/or cell sample, the prostate tissue and/or cell sample is determined as a healthy prostate tissue and/or cell sample; and
if the imprinted gene Z18, Z19, Z20, Z21, Z25, Z26, Z27, or Z28 is expressed as having a loss of imprinting and/or a copy number variation in the cell nuclei in the prostate tissue and/or cell sample, the prostate tissue and/or cell sample is determined as a prostate cancer tissue and/or cell sample;
or
(i) subjecting a gastric tissue and/or cell sample to an analysis of the expression of any one, or a combination of at least two, of the imprinted genes Z18, Z19, Z20, Z21, Z22, Z26, and Z27 so that:
if the imprinted gene Z18, Z19, Z20, Z21, Z22, Z26, or Z27 is not expressed or is expressed as normal in cell nuclei in the gastric tissue and/or cell sample, the gastric tissue and/or cell sample is determined as a healthy gastric tissue and/or cell sample; and
if the imprinted gene Z18, Z19, Z20, Z21, Z22, Z26, or Z27 is expressed as having a loss of imprinting and/or a copy number variation in the cell nuclei in the gastric tissue and/or cell sample, the gastric tissue and/or cell sample is determined as a gastric carcinoma tissue and/or cell sample;
or
(j) subjecting an esophageal tissue and/or cell sample to an analysis of the expression of any one, or a combination of at least two, of the imprinted genes Z18, Z19, Z21, Z22, Z26, Z27, and Z28 so that:
if the imprinted gene Z18, Z19, Z21, Z22, Z26, Z27, or Z28 is not expressed or is expressed as normal in cell nuclei in the esophageal tissue and/or cell sample, the esophageal tissue and/or cell sample is determined as a healthy esophageal tissue and/or cell sample; and
if the imprinted gene Z18, Z19, Z21, Z22, Z26, Z27, or Z28 is expressed as having a loss of imprinting and/or a copy number variation in the cell nuclei in the esophageal tissue and/or cell sample, the esophageal tissue and/or cell sample is determined as an esophageal cancer tissue and/or cell sample;
or
(k) subjecting a mammary tissue and/or cell sample to an analysis of the expression of any one, or a combination of at least two, of the imprinted genes Z18, Z19, Z21, Z22, and Z27 so that:
if the imprinted gene Z18, Z19, Z21, Z22, or Z27 is not expressed or is expressed as normal in cell nuclei in the mammary tissue and/or cell sample, the mammary tissue and/or cell sample is determined as a healthy mammary tissue and/or cell sample; and
if the imprinted gene Z18, Z19, Z21, Z22, or Z27 is expressed as having a loss of imprinting and/or a copy number variation in the cell nuclei in the mammary tissue and/or cell sample, the mammary tissue and/or cell sample is determined as a mammary cancer tissue and/or cell sample;
or
(l) subjecting a thyroid tissue and/or cell sample to an analysis of the expression of any one, or a combination of at least two, of the imprinted genes Z18, Z19, Z20, Z21, and Z23 so that:
if the imprinted gene Z18, Z19, Z20, Z21, or Z23 is not expressed or is expressed as normal in cell nuclei in the thyroid tissue and/or cell sample, the thyroid tissue and/or cell sample is determined as a healthy thyroid tissue and/or cell sample; and
if the imprinted gene Z18, Z19, Z20, Z21, or Z23 is expressed as having a loss of imprinting and/or a copy number variation in the cell nuclei in the thyroid tissue and/or cell sample, the thyroid tissue and/or cell sample is determined as a thyroid cancer tissue and/or cell sample;
or
(m) subjecting a bladder tissue and/or cell sample to an analysis of the expression of any one, or a combination of at least two, of the imprinted genes Z18, Z19, Z20, Z21, Z22, Z26, Z27, and Z28 so that:
if the imprinted gene Z18, Z19, Z20, Z21, Z22, Z26, Z27, or Z28 is not expressed or is expressed as normal in cell nuclei in the bladder tissue and/or cell sample, the bladder tissue and/or cell sample is determined as a healthy bladder tissue and/or cell sample; and
if the imprinted gene Z18, Z19, Z20, Z21, Z22, Z26, Z27, or Z28 is expressed as having a loss of imprinting and/or a copy number variation in the cell nuclei in the bladder tissue and/or cell sample, the bladder tissue and/or cell sample is determined as a bladder cancer tissue and/or cell sample;
or
(n) subjecting a liver tissue and/or cell sample to an analysis of the expression of any one, or a combination of at least two, of the imprinted genes Z17, Z19, Z21, Z22, Z26, Z27, and Z28 so that:
if the imprinted gene Z17, Z19, Z21, Z22, Z26, Z27, or Z28 is not expressed or is expressed as normal in cell nuclei in the liver tissue and/or cell sample, the liver tissue and/or cell sample is determined as a healthy liver tissue and/or cell sample; and
if the imprinted gene Z17, Z19, Z21, Z22, Z26, Z27, or Z28 is expressed as having a loss of imprinting and/or a copy number variation in the cell nuclei in the liver tissue and/or cell sample, the liver tissue and/or cell sample is determined as a liver cancer tissue and/or cell sample;
or
(o) subjecting a pancreatic tissue and/or cell sample to an analysis of the expression of any one, or a combination of at least two, of the imprinted genes Z17, Z18, Z19, Z20, Z21, Z22, Z26, Z27, and Z28 so that:
if the imprinted gene Z17, Z18, Z19, Z20, Z21, Z22, Z26, Z27, or Z28 is not expressed or is expressed as normal in cell nuclei in the pancreatic tissue and/or cell sample, the pancreatic tissue and/or cell sample is determined as a healthy pancreatic tissue and/or cell sample; and
if the imprinted gene Z17, Z18, Z19, Z20, Z21, Z22, Z26, Z27, or Z28 is expressed as having a loss of imprinting and/or a copy number variation in the cell nuclei in the pancreatic tissue and/or cell sample, the pancreatic tissue and/or cell sample is determined as a pancreatic cancer tissue and/or cell sample;
or
(p) subjecting a lymphatic tissue and/or cell sample to an analysis of the expression of any one, or a combination of at least two, of the imprinted genes Z18, Z19, Z21, Z22, Z26, Z27, and Z28 so that:
if the imprinted gene Z18, Z19, Z21, Z22, Z26, Z27, or Z28 is not expressed or is expressed as normal in cell nuclei in the lymphatic tissue and/or cell sample, the lymphatic tissue and/or cell sample is determined as a healthy lymphatic tissue and/or cell sample; and
if the imprinted gene Z18, Z19, Z21, Z22, Z26, Z27, or Z28 is expressed as having a loss of imprinting and/or a copy number variation in the cell nuclei in the lymphatic tissue and/or cell sample, the lymphatic tissue and/or cell sample is determined as a lymphoma tissue and/or cell sample;
or
(q) subjecting a colorectal tissue and/or cell sample to an analysis of the expression of any one, or a combination of at least two, of the imprinted genes Z19, Z20, Z21, Z22, Z26, and Z27 so that:
if the imprinted gene Z19, Z20, Z21, Z22, Z26, or Z27 is not expressed or is expressed as normal in cell nuclei in the colorectal tissue and/or cell sample, the colorectal tissue and/or cell sample is determined as a healthy colorectal tissue and/or cell sample; and
if the imprinted gene Z19, Z20, Z21, Z22, Z26, or Z27 is expressed as having a loss of imprinting and/or a copy number variation in the cell nuclei in the colorectal tissue and/or cell sample, the colorectal tissue and/or cell sample is determined as a colorectal cancer tissue and/or cell sample;
or
(r) subjecting a skin tissue and/or cell sample to an analysis of the expression of any one, or a combination of at least two, of the imprinted genes Z18, Z19, Z21, Z22, Z25, and Z28 so that:
if the imprinted gene Z18, Z19, Z21, Z22, Z25, or Z28 is not expressed or is expressed as normal in cell nuclei in the skin tissue and/or cell sample, the skin tissue and/or cell sample is determined as a healthy skin tissue and/or cell sample; and
if the imprinted gene Z18, Z19, Z21, Z22, Z25, or Z28 is expressed as having a loss of imprinting and/or a copy number variation in the cell nuclei in the skin tissue and/or cell sample, the skin tissue and/or cell sample is determined as a skin cancer tissue and/or cell sample;
or
(s) subjecting a lung tissue and/or cell sample to an analysis of the expression of any one, or a combination of at least two, of the imprinted genes Z18, Z19, Z21, Z22, Z25, Z26, Z27, and Z28 so that:
if the imprinted gene Z18, Z19, Z21, Z22, Z25, Z26, Z27, or Z28 is not expressed or is expressed as normal in cell nuclei in the lung tissue and/or cell sample, the lung tissue and/or cell sample is determined as a healthy lung tissue and/or cell sample; and
if the imprinted gene Z18, Z19, Z21, Z22, Z25, Z26, Z27, or Z28 is expressed as having a loss of imprinting and/or a copy number variation in the cell nuclei in the lung tissue and/or cell sample, the lung tissue and/or cell sample is determined as a lung cancer tissue and/or cell sample.

15. A use of the tumor marker of any of claims 1 to 5, of the tumor diagnosis model of any of claims 6 to 8, or of the tumor diagnosis device of any of claims 9 to 14 in preparing a drug and/or a reagent for use in diagnosing, screening, or treating a tumor.
